(19) United States
(12) Patent Application Publication
(10) Pub. No.: US 2023/0347902 A1
(43) Pub. Date: Nov. 2, 2023

(54) DATA PROCESSING SYSTEM, DRIVER ASSISTANCE SYSTEM, DATA PROCESSING DEVICE, AND WEARABLE DEVICE

(71) Applicant: SEMICONDUCTOR ENERGY LABORATORY CO., LTD., Atsugi-shi, Kanagawa-ken (JP)

(72) Inventors: Shunpei YAMAZAKI, Setagaya, Tokyo (JP); Takayuki IKEDA, Atsugi, Kanagawa (JP)

(21) Appl. No.: 17/791,345

(22) PCT Filed: Jan. 12, 2021
(86) PCT No.: PCT/IB2021/050183

§ 371 Date: Jul. 7, 2022

(30) Foreign Application Priority Data
Jan. 22, 2020 (JP) 2020-008647

(51) Int. Cl. B60W 40/08; A61B 5/18; B60W 50/14

(52) U.S. Cl. CPC B60W 40/08; A61B 5/18; B60W 50/14; B60W 2540/22; B60W 2540/221; B60W 2050/146; B60W 2540/225; B60W 2420/42

(57) ABSTRACT

A novel data processing device is provided. The data processing device includes a conversation data generation unit, an image processing unit, a display device, an imaging device, an operation unit, a biosensor, a speaker, and a microphone. The conversation data generation unit includes a classifier that has learned preference information of a user, and the biosensor detects the biological information of the user who wears the data processing device. The imaging device captures a first image. When a designated first object is detected in the first image, the operation unit generates a second image where a second object overlaps with part of the first object. The image processing unit displays the second image on the display device. The conversation data generation unit generates first conversation data based on the biological information and the preference information, and outputs the first conversation data from the speaker. The microphone obtains second conversation data corresponding to a response from the user and outputs the second conversation data to the classifier. The classifier has a function of updating the preference information with the use of the second conversation data.

### TECHNICAL FIELD

One embodiment of the present invention relates to a data processing device or a wearable device that improves a user's action, decision-making, and the user's safety with an object created utilizing a computer to converse with the user. One embodiment of the present invention also relates to an electronic device including a data processing device. One embodiment of the present invention also relates to a data processing system or a driver assistance system that uses the data processing device.

### BACKGROUND ART

It is known that a human being placed in an environment where the movement is restricted for a long time suffers from physical stress and mental stress, and becomes distracted, drowsy, and excessively sensitive to minute changes. In other words, it is known that human being feels physical and mental stress when held in an environment where the movement is restricted for a long time.

In the case where a user (hereinafter, a driver) drives a means of transportation (an object that moves while carrying human being or things), for example, the driver who is driving the means of transportation is restricted in movement and range of eyesight, and thus is subjected to the above-mentioned stress.

Autonomous driving of vehicles has been developed in recent years, and control technology that enables autonomous driving of vehicles has been steadily improving. However, it is predicted that improvements in law, environment, equipment, and the like still need more time before all the vehicles on the road become autonomously controlled. Note that in the description of this specification, a vehicle (a means of transportation with wheels) is used as an example of a means of transportation. Note that a means of transportation can include a train, a ship, an airplane, or the like.

Patent Document 1 discloses a system and a method that respond to the behavior (drowsiness) of a driver. A system that turns on an automatic braking system when detecting drowsiness of the driver is disclosed, for example.

### REFERENCE [Patent Document]

[Patent Document 1] Japanese Published Patent Application No. 2017-200822

### SUMMARY OF THE INVENTION

### Problems to be Solved by the Invention

To reduce stress caused by continuous driving on an expressway, technology of not only autonomous driving control but also semi-autonomous driving control has been developed. Semi-autonomous driving frees a driver from continuous stress of high-speed driving. However, semi-autonomous driving entails the time when autonomous driving switches to the driver's driving, and also sometimes requires immediate actions in occasions such as a collision between vehicles or a pedestrian dashing out into the road. Thus, even with semi-autonomous driving control, there remains an issue where the driver is still held in a restricting environment and loses his/her attention because of drowsiness or the like.

There is also an attempt to alert a driver with the use of a warning beep or flashing light when decrease in a driver's attention caused by drowsiness or the like is detected. However, even if the driver is alerted with the use of an warning beep, flashing light, or the like, the driver gets used to the same kind of warning beep or flashing light, so that the warning beep or flashing light ceases to function as a means of alerting a driver.

In view of the above problems, an object of one embodiment of the present invention is to provide a data processing device that activates consciousness by conversation or the like. Another object of one embodiment of the present invention is to provide a data processing device that generates conversation data. Another object of one embodiment of the present invention is to provide a data processing device having an augmented reality function that associates conversation data with the movement of an object. Another object of one embodiment of the present invention is to provide a data processing device that generates conversation data with the use of a classifier including preference information of a user. Another object of one embodiment of the present invention is to provide a data processing device that generates conversation data with the use of biological information detected by a biosensor and preference information included in a classifier. Another object of one embodiment of the present invention is to provide a data processing device in which preference information included in a classifier is updated with the use of biological information of a user detected by a biosensor and the user's conversation data.

Note that the description of these objects does not preclude the existence of other objects. One embodiment of the present invention does not have to achieve all these objects. Other objects are apparent from the description of the specification, the drawings, the claims, and the like, and other objects can be derived from the description of the specification, the drawings, the claims, and the like.

### Means for Solving the Problems

One embodiment of the present invention is a data processing system including a biosensor, a conversation data generation unit, an operation unit, a speaker, and a microphone. The conversation data generation unit includes a classifier that has learned first information of a user. The biosensor is capable of detecting second information of the user. The conversation data generation unit is capable of generating first conversation data based on the first information and the second information. The speaker outputs the first conversation data, and the microphone is capable of obtaining second conversation data from the user and outputting the second conversation data to the classifier. The classifier is capable of updating the first information with use of the second conversation data.

One embodiment of the present invention is a driver assistance system including a biosensor, a conversation data generation unit, an operation unit, a speaker, and a microphone. The conversation data generation unit includes a classifier that has learned first information of a driver. The biosensor is capable of detecting second information of the driver. The conversation data generation unit is capable of generating first conversation data based on the first information and the second information. The speaker outputs the first conversation data, and the microphone is capable of obtaining second conversation data from the driver and outputting the second conversation data to the classifier. The classifier is capable of updating the first information with use of the second conversation data.

One embodiment of the present invention is a data processing device including a conversation data generation unit, an operation unit, a biosensor, a speaker, and a microphone. The conversation data generation unit includes a classifier that learns first information of a user, and the biosensor has a function of detecting second information of the user who uses the data processing device. Note that a classifier that has learned the first information of the user may be used as the classifier. The conversation data generation unit has a function of generating first conversation data based on the first information and the second information, and the speaker has a function of outputting the first conversation data. The microphone has a function of obtaining second conversation data corresponding to a response from the user and outputting the second conversation data to the classifier, and the classifier has a function of updating the first information with use of the second conversation data.

One embodiment of the present invention is a data processing device including a conversation data generation unit, an operation unit, an image processing unit, a display device, an imaging device, a biosensor, a speaker, and a microphone. The conversation data generation unit includes a classifier that learns first information of a user, and the biosensor has a function of detecting second information of the user who uses the data processing device. Note that a classifier that has learned the first information of the user may be used as the classifier. The imaging device has a function of capturing a first image, and the operation unit has a function of detecting a designated first object in the first image. The image processing unit has a function of generating a second image where a second object overlaps with part of the first object when the first object is detected, and the image processing unit has a function of displaying the second image on the display device. The conversation data generation unit has a function of generating first conversation data based on the first information and the second information, and the speaker has a function of outputting the first conversation data in conjunction with movement of the second object. The microphone has a function of obtaining second conversation data corresponding to a response from the user and outputting the second conversation data to the classifier, and the classifier has a function of updating the first information with use of the second conversation data.

One embodiment of the present invention is a data processing device including a conversation data generation unit, an image processing unit, a display device, an imaging device, an operation unit, a biosensor, a speaker, and a microphone. The conversation data generation unit is supplied with first information of a user, and the biosensor has a function of detecting second information of the user who uses the data processing device. The imaging device has a function of capturing a first image, and the operation unit has a function of detecting a designated first object in the first image. The image processing unit has a function of generating a second image where a second object overlaps with part of the first object when the first object is detected, and the image processing unit has a function of displaying the second image on the display device. The conversation data generation unit has a function of generating first conversation data based on the first information and the second information, and the speaker has a function of outputting the first conversation data in conjunction with movement of the second object. The microphone has a function of obtaining second conversation data corresponding to a response from the user. The conversation data generation unit has a function of outputting the second conversation data.

In any of the above structures, the first information is preferably preference information. The second information is preferably biological information.

In any of the above structures, the data processing device is preferably a wearable device having a function of glasses. Furthermore, a wearable device that allows the location where the second object is displayed to be specified is preferable. In addition, the data processing device preferably includes setting information for setting the location where the second object is displayed to a passenger seat of a car or the like.

### Effect of the Invention

According to one embodiment of the present invention, a data processing device that activates consciousness by conversation or the like can be provided. According to one embodiment of the present invention, a data processing device that generates conversation data can be provided. According to one embodiment of the present invention, a data processing device having an augmented reality function that associates conversation data with the movement of an object can be provided. According to one embodiment of the present invention, a data processing device that generates conversation data with the use of a classifier including preference information of a user can be provided. According to one embodiment of the present invention, a data processing device that generates conversation data with the use of biological information detected by a biosensor and preference information included in a classifier can be provided. According to one embodiment of the present invention, a data processing device in which preference information included in a classifier is updated with the use of biological information of a user detected by a biosensor and the user's conversation data.

Note that the effects of embodiments of the present invention are not limited to the effects listed above. The effects listed above do not preclude the existence of other effects. The other effects are effects that are not described in this section and will be described below. The other effects not described in this section will be apparent from and can be derived as appropriate from the descriptions of the specification, the drawings, and the like by those skilled in the art. One embodiment of the present invention has at least one effect of the effects listed above and/or the other effects. Accordingly, depending on the case, one embodiment of the present invention does not have the effects listed above in some cases.

### BRIEF DESCRIPTION OF THE DRAWINGS

FIG. 1A is a drawing illustrating a case where a car interior (a passenger seat) is seen from a driver seat. FIG. 1B and FIG. 1C are drawings illustrating data processing devices. FIG. 1D is a drawing illustrating a case where a car interior is seen through a wearable device.

FIG. 2 is a flow chart showing the operation of a wearable device.

FIG. 3 is a flow chart showing the operation of a wearable device.

FIG. 4 is a block diagram illustrating a wearable device and a vehicle.

FIG. 5A is a block diagram illustrating a wearable device. FIG. 5B is a block diagram illustrating a vehicle.

FIG. 6A and FIG. 6B are drawings illustrating a configuration example of a wearable device.

FIG. 7A and FIG. 7B are drawings each illustrating a configuration example in which an object is seen through a data processing device.

FIG. 8A is a perspective view illustrating an example of a semiconductor wafer, FIG. 8B is a perspective view illustrating an example of a chip, and FIG. 8C and FIG. 8D are perspective views illustrating examples of electronic components.

FIG. 9 is a block diagram illustrating a CPU.

FIG. 10A and FIG. 10B are perspective views illustrating a semiconductor device.

FIG. 11A and FIG. 11B are perspective views illustrating a semiconductor device.

FIG. 12A and FIG. 12B are perspective views illustrating a semiconductor device.

FIG. 13A and FIG. 13B are drawings each showing a hierarchy of a variety of memory devices.

FIG. 14A to FIG. 14F are each a perspective view or a schematic view illustrating an example of an electronic device including a data processing device.

FIG. 15A to FIG. 15E are each a perspective view or a schematic view illustrating an example of an electronic device including a data processing device.

### MODE FOR CARRYING OUT THE INVENTION

Embodiments will be described in detail with reference to the drawings. However, the present invention is not limited to the following description, and it is readily appreciated by those skilled in the art that modes and details can be modified in various ways without departing from the spirit and the scope of the present invention. Thus, the present invention should not be interpreted as being limited to the description of embodiments below.

Note that in structures of the present invention described below, the same reference numerals are used in common for the same portions or portions having similar functions in different drawings, and a repeated description thereof is omitted. Furthermore, the same hatch pattern is used for the portions having similar functions, and the portions are not denoted by reference numerals in some cases.

The position, size, range, or the like of each component illustrated in drawings does not represent the actual position, size, range, or the like in some cases for easy understanding. Therefore, the disclosed invention is not necessarily limited to the position, size, range, or the like disclosed in the drawings.

### Embodiment 1

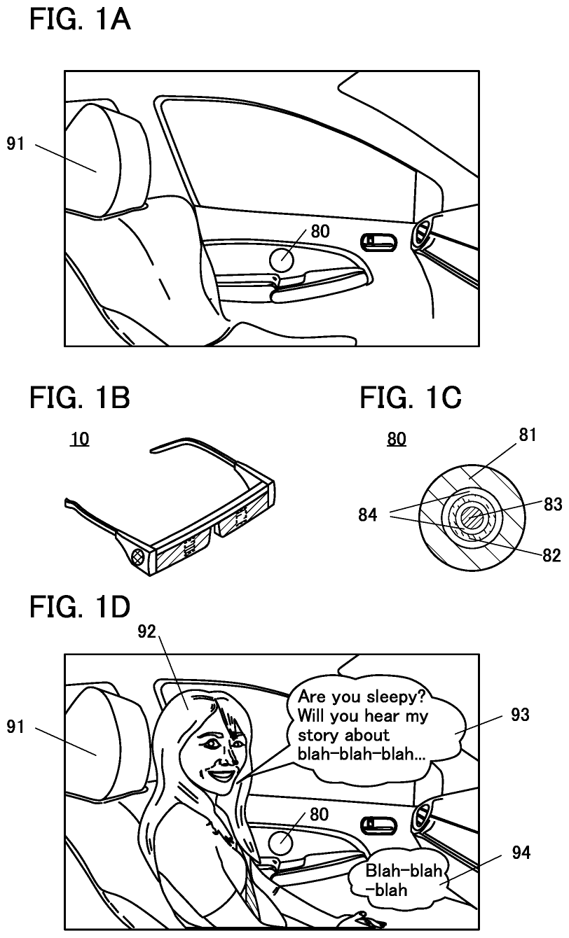

In this embodiment, a data processing device will be described with reference to FIG. 1A to FIG. 7B.

A data processing device of one embodiment of the present invention is preferably a wearable device, a portable information terminal, an automatic voice response device, a stationary electronic device, or an embedded electronic device. A wearable device of one embodiment of the present invention includes a display device having an eyeglasses function, as an example. The wearable device includes a display device capable of displaying an image of a created object superimposed on an image seen through the eyeglasses function. Note that displaying an image of a created object superimposed on an image seen through the eyeglasses function can be referred to as augmented reality (AR) or mixed reality (MR).

The wearable device includes a conversation data generation unit, an operation unit, an image processing unit, a display device, an imaging device, a biosensor, a speaker, and a microphone. In the case where the data processing device of one embodiment of the invention is a stationary or embedded electronic device, the electronic device preferably includes at least a conversation data generation unit, an operation unit, a biosensor, a speaker, and a microphone.

The conversation data generation unit includes a classifier that has learned the user preference information. As the classifier, a classifier prepared in a server computer on the cloud can be used. When the user preference information is learned on the cloud, power consumption of the wearable device and the number of components such as a memory can be reduced. In addition, the use of the classifier on the cloud enables usage history of the data processing device used by the user (e.g., titles of DVDs that have been played, viewing history of TV programs, list of items stored in a refrigerator, or operation history of a dish washer, in the case where the data processing device is incorporated in consumer electronics or the like) to be learned as the preference information by the classifier. Note that the preference information of one embodiment of the present invention can be used as a combination of one or more pieces of preference information.

The biosensor is capable of detecting biological information of a user who wears the wearable device. The biological information preferably includes one or more of the following: body temperature, blood pressure, pulse rate, sweating rate, blood sugar level, red blood cell count, respiratory rate, eye water content, eye blinking count, and the like. Note that the biological information of one embodiment of the present invention can be used as a combination of one or more pieces of biological information.

The imaging device includes a first imaging device and a second imaging device. The first imaging device captures a first image in the user's eye direction. The second imaging device captures a second image for detecting the movement of the user's eyes, how wide the eyelids open, eye blinking count, and the like. The number of imaging devices is not limited, and may be three or more.

The operation unit is capable of image analysis. The image analysis can utilize a convolutional neural network (hereinafter, CNN), as an example. The use of CNN enables a designated first object to be detected in the first image. The image processing unit creates a third image such that a second object overlaps with part of the first object when the first object is detected, and the image processing unit is capable of displaying the third image on the display device.

The image analysis method is not limited to CNN. As image analysis methods different from CNN, R-CNN (Regions with Convolutional Neural Networks), YOLO (You Only Look Once), SSD (Single Shot MultiBox Detector), and the like can be used. A method called semantic segmentation using neural networks can also be used. As the semantic segmentation method, FCN (Fully Convolutional Network), SegNet, U-Net, PSPNet (Pyramid Scene Parsing Network), and the like can be used.

Based on the second image, specified eye movements (e.g., the movement of eyeballs), movements around eyes such as eyelid movement (hereinafter described as eye movement to simplify the description), and the like can be detected. By detecting eye movements, movement of the line of sight can be detected. By using the analysis result of the first image, the direction of the user's face can be detected.

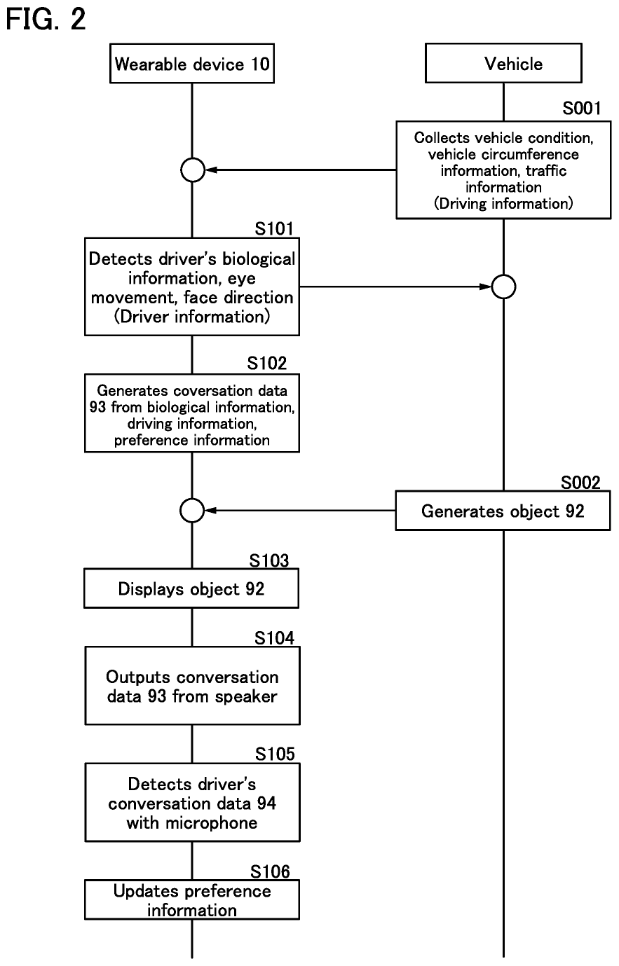

The conversation data generation unit is capable of generating first conversation data based on the biological information and preference information. The speaker is capable of outputting the first conversation data. It is preferable that the first conversation data be output in conjunction with the movement of the second object. The microphone is capable of acquiring second conversation data corresponding to the user's response and converting it to language data. The language data is supplied to the classifier. The classifier is capable of updating the preference information with the use of the language data. Furthermore, the conversation data generation unit is capable of generating conversation data where the preference information and other information are combined. Examples of other information include the car driving information, vehicle information, driver information, information acquired by an in-car imaging device, and information obtained through the Internet. Other information will be described in detail with reference to FIG. 2. The conversation data preferably contains a self-counseling function.

An image of the passenger seat of the car, for example, can be registered as the first object. The image may be registered freely by the user, or a subject image may be pre-registered in the wearable device. In the case where a glasses-like wearable device worn by the user detects a passenger seat in the first image, for example, the second object or the like can be displayed in a position overlapping with the passenger seat in the first image. Note that the type of the second object is not limited. A person, an animal, or the like extracted from a photo or a moving image can be registered as the second object. It may also be an object or illustration downloaded from the other contents. Alternatively, it may be an originally created object. It is preferable that the second object be a person who could relax someone's mind or atmosphere. Thus, the wearable device of one embodiment of the present invention enables brain activation to be facilitated through conversation with the registered object, thereby reducing the effect of stress or the like. Note that the second object can also be referred to as a character.

Thus, one embodiment of the present invention can be referred to as a data processing system or autonomous driving assistance system using the above-descried data processing device.

Processing details of the above-described wearable device will be described with reference to FIG. 1A to FIG. 1D. In FIG. 1A, an image of a passenger seat of a car is registered as an object 91, for example. An automatic voice response device 80, which will be described later, is provided on a door by the passenger seat.

FIG. 1A illustrates a case where the car interior (the passenger seat) is seen from the driver seat. It can be seen from FIG. 1A that nobody is sitting on the passenger seat.

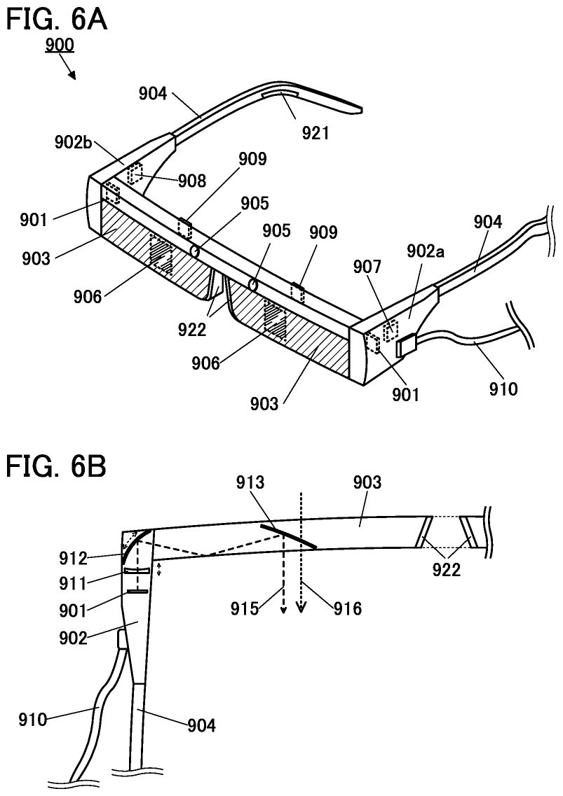

FIG. 1B and FIG. 1C illustrate the data processing devices described in this embodiment. The data processing device shown in FIG. 1B is a wearable device 10. The wearable device 10 will be described in detail with reference to FIG. 6A and FIG. 6B.

The data processing device shown in FIG. 1C is an automatic voice response device 80 with a biosensor. The automatic voice response device 80 may also be referred to as an AI speaker. The automatic voice response device 80 includes a speaker 81, a microphone 82, and a biosensor 83. Although not illustrated in FIG. 1C, the automatic voice response device 80 may include the conversation data generation unit and the operation unit, in addition to the speaker 81, the microphone 82, and the biosensor 83. With parts of a housing 84 of the automatic voice response device 80, the speaker 81, the microphone 82, and the biosensor 83 can be separated from one another. Note that the speaker 81, the microphone 82, and the biosensor 83 do not have to be separated by the housing 84.

FIG. 1D illustrates a case where the car interior is seen through the wearable device 10, as an example. The first imaging device is capable of capturing an image of the car interior as a first image. The operation unit is capable of detecting, with the use of CNN or the like, the position of the passenger seat registered as the object 91 in the first image. The image processing unit is capable of displaying an image of a woman registered as an object 92 such that the image overlaps with the position where the object 91 is detected. It is preferable that the automatic voice response device 80 be configured to operate in the case where the user (hereinafter, a driver) does not use the wearable device 10.

The biosensor is capable of detecting the biological information of the driver. The conversation data generation unit is capable of selecting the detected biological information and the preference information from the classifier in the conversation data generation unit, and generating conversation data 93 by combining the biological information and the preference information. The preference information may be selected from a category with a large number of registered items or from a category with a small number of registered items. When the preference information is selected from the category with a large number of registered items, the driver's brain is activated through thinking about information of interest. However, there is also a case where the driver's brain is activated through retrospection when the preference information is selected from the category with a small number of registered items. It is preferable that the preference information be determined by being combined with biological information.

Here, a case where the biosensor detects the drowsiness of the driver will be described. The biosensor can determine that the drowsiness of the driver is increasing when the driver's heart rate is decreasing with driving time. However, the driver's heart rate tends to increase while he/she is driving a car. The biosensor is capable of detecting a change in heart rate by monitoring on a regular basis the heartbeat intervals of the driver while driving.

An infrared sensor, as an example, can be used as the biosensor. For the wearable device 10 in the form of glasses, the biosensor is preferably placed on a pad contacting the nose, or on a temple touching the ear. For detecting the drowsiness, the number of blinking times or the like can be added to determination conditions. Capable of detecting the movement of the user's eyes, how wide the eyelids open, or the like, the second imaging device can be included as one type of the biosensor. In the case where the biosensor does not contact the body, the biosensor preferably monitors the temple area.

In FIG. 1D, as a result of the biosensor detecting the driver's drowsiness, the conversation data 93, a question “Are you sleepy?” to the driver, is generated and asked through the speaker. This corresponds to an alert or warning to the driver based on the biosensor. In addition, the conversation data generation unit generates, as the conversation data 93, conversation data concerning “blah-blah-blah”, which is extracted from the preference information, in order to stimulate the driver's brain. At that time, it is preferable that the type of voice, the tone of voice, the speed of conversation, or the like that matches the registered object 92 be selected in accordance with the strength of a stimulus to be provided to the driver's brain. For example, the object 92 asks a question, which is the conversation data 93 “Will you hear my story about blah-blah-blah,” through the speaker. When the object 92 moves in conjunction with the conversation data 93, a sense of intimacy with the object 92 is likely to be provided. The conversation data 93 to be generated is preferably conversation data in the form of question that requires a response, in which case the driver's brain can be activated by the need of a response. In the case where the microphone of the wearable device 10 detects the driver's voice (conversation data 94), the conversation data 94 is converted into language data in the conversation data generation unit, and the language data can update the preference information.

FIG. 2 is a flow chart describing the operation of the wearable device 10. The flow chart in FIG. 2 as an example shows the relation between the wearable device 10 and a vehicle. Each operation will be described as a step, with reference to FIG. 2.

Step S001 is a step in which a monitoring unit in the vehicle collects driving information such as the vehicle condition and the vehicle circumference information. The monitoring unit may be referred to as an engine control unit. The engine control unit is capable of controlling the engine's condition by means of computer control or controlling driving with the use of a plurality of sensors. In addition, the vehicle collects traffic information or the like through a satellite or wireless communication. Note that the vehicle can supply the wearable device 10 with the driving information.

Step S101 is a step in which the wearable device 10 detects the biological information of the driver, detects the eye movement or face direction of the driver with the use of the first image seen by the driver and the second image, and supplies the first image, the second image, and the driver information (the biological information of the driver, the eye movement or face direction of the driver, and the like) to the vehicle. The vehicle can turn on an automatic braking system, automatic tracking drive, or the like with the use of the driver information, thereby enabling semi-autonomous driving or autonomous driving. Thus, with the driver information detected by the wearable device 10 being given to the vehicle, car accidents caused by distracted driving or drowsy driving can be prevented. The semi-autonomous driving or autonomous driving can be canceled based on the driver information. The driver information is also given to the conversation data generation unit.

Step S102 is a step in which the conversation data generation unit generates the conversation data 93, using the driving information, the driver information including the biological information, and the preference information included in the classifier. It is preferable that the conversation data 93 that corresponds to an alert or warning be generated based on the biological information. As an example, the conversation data 93 related to the health can be generated for “blah-blah-blah”, using the biological information. Alternatively, the conversation data 93 where the temperature inside the car and the biological information are combined can be generated for “blah-blah-blah”, using the driving information. Alternatively, the conversation data 93 on refueling time or the like can be generated for “blah-blah-blah”, using the driving information. Alternatively, for “blah-blah-blah”, the conversation data 93 can be generated using the preference information such as music, a TV program, food, a recently-taken photo, or usage history of consumer electronics such as the contents of the refrigerator. Note that the conversation data generation unit preferably generates the conversation data 93 in a question form that requires a response from the driver.

Step S002 is a step of generating the object 92. When the object 91 (the passenger seat of the car) is detected in the first image taken by the wearable device 10, the object 92 (an image of a woman) that overlaps with the object 91 is generated. Note that the object 92 is preferably configured to reflect the position information of the object 91 that is detected in the first image. For example, in the case where the object 91 is detected at the center of the first image, the object 92 is generated at the center so as to overlap with the object 91, as illustrated in FIG. 1D. Note that the object 92 preferably has a direction in the same way as the case where a person sits in the passenger seat. As another example, in the case where the object 91 is detected in a small size at the edge of the first image, the object 92 is generated in a position that takes the position relation of the detected object 91 into account. In other words, the object 92 corresponds to a person who sits in the passenger seat at the edge of the glasses. Thus, Step S102 is processed in the wearable device 10 and Step S002 is processed in the vehicle, allowing the simultaneous processing of Step S102 and Step S002.

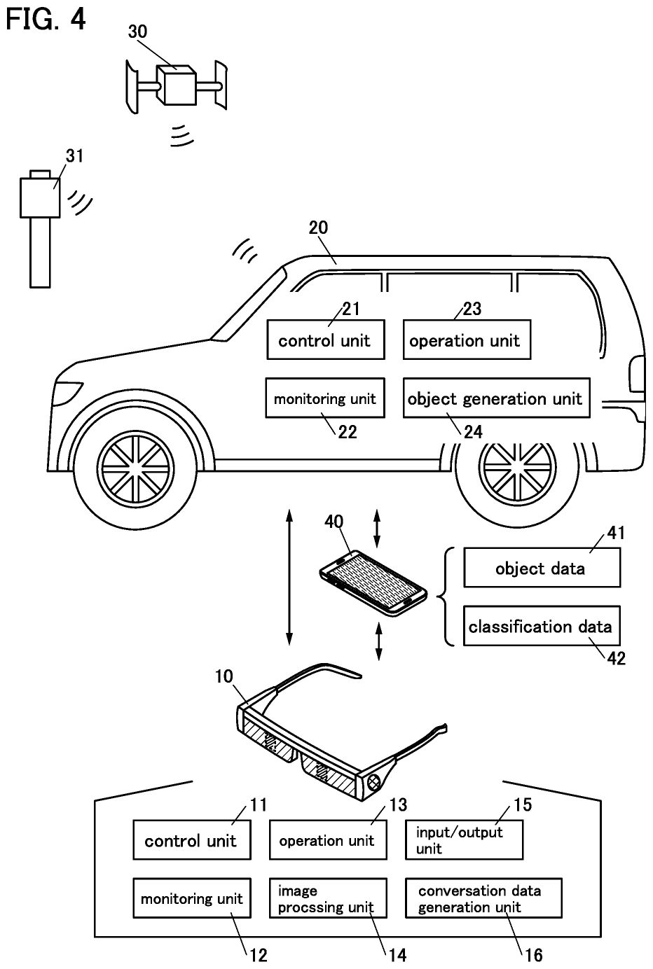

One embodiment of the present invention shows an example in which the object 92 is generated with the use of the object generation unit in the vehicle. However, the object generation unit may be included in the wearable device 10. Alternatively, the object 92 can be generated with the use of the object generation unit prepared in a server computer on the cloud. Alternatively, a configuration where a portable accelerator that can be carried around includes a memory device that stores the object 92 and the object generation unit is also possible. Note that the relation between the wearable device 10 and the vehicle will be described in detail with reference to FIG. 4.

Step S103 is a step in which the object 92 is displayed, overlapping with the object 91. When the object 92 is superimposed on an image seen by the glasses function of the wearable device 10, extended reality or mixed reality can be created. Thus, through the wearable device 10, the object 92 shown in FIG. 1D can be displayed.

Step S104 is a step in which the conversation data 93 is output from the speaker in accordance with the display of the object 92. It is preferable that the object 92 move in conjunction with the conversation data 93. In that case, it is preferable that the type of voice, the tone of voice, the speed of conversation, or the like change in accordance with the movement of the object 92. The intensity of stimulus given to the driver's brain is changed in accordance with the changeable movement with gestures and voice of the object 92. Note that the effect of the stimulus given to the driver's brain can be observed as the amount of change detected by the biosensor. It is also possible to update the preference information with the use of the amount of change.

Step S105 is a step in which the conversation data 94 including the driver's reply to the conversation data 93 is detected by the microphone.

In Step S106, the conversation data 94 detected by the wearable device 10 is converted into language data in the conversation data generation unit, enabling the preference information to be updated with the use of the language data. Thus, the classifier in the wearable device 10 can learn what kind of preference information activates the driver's brain through learning of the conversation data 93 and the conversation data 94 between the wearable device 10 and the driver, and update the weight coefficient. The conversation data generation unit is capable of learning the movement of the object 92 displayed by the wearable device 10, and the type of voice, the tone of voice, the speed of conversation and the like output in accordance with the movement of the object 92.

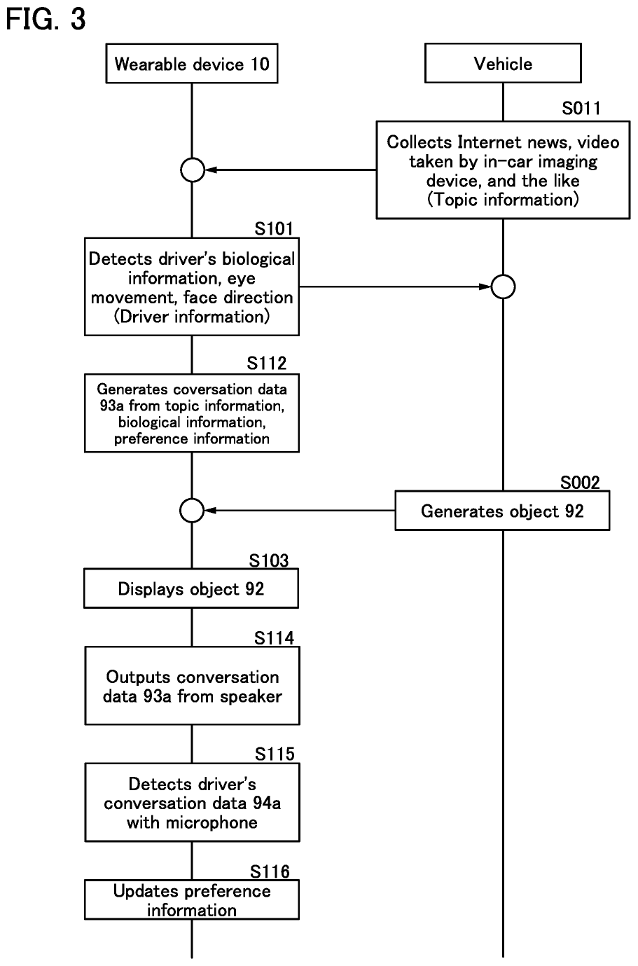

FIG. 3 is a flow chart describing the operation of the wearable device 10, which is different from FIG. 2. Steps different from FIG. 2 will be described; for steps involving the same processes as those of FIG. 2, description for FIG. 2 is referred to and detailed description will be omitted here.

In Step S011, the Internet news obtained through a satellite or wireless communication by the control unit of the vehicle can be collected as topic information. In addition, the in-car imaging device in the monitoring unit of the vehicle is capable of collecting videos taken by the driving vehicle. For example, the kind and speed of a vehicle that has gone by, the outfit of a pedestrian, a video of an abnormally-driven vehicle, or the like can be collected as topic information. The vehicle can supply the wearable device 10 with the topic information.

Step S112 is a step in which the conversation data generation unit generates conversation data 93a with the use of the topic information, the biological information, and the preference information included in the classifier. It is preferable that the classifier extract information that is highly likely to activate the driver's brain, from the topic information. Note that it is preferable that the conversation data 93a that corresponds to an alert or warning be generated from the biological information. As an example, the conversation data 93a is generated with the use of information that is highly likely to activate the driver's brain with the topic information. Note that it is preferable that the conversation data generation unit generate the conversation data 93a in a question form that requires a reply from the driver.

Step S114 is a step in which the conversation data 93a is output from the speaker in accordance with the display of the object 92. Note that it is preferable that the object 92 move in conjunction with the conversation data 93a. In that case, it is preferable that the type of voice, the tone of voice, the speed of conversation, or the like change in accordance with the movement of the object 92. Since the topic information is preference information that is high in degree of preference, changes in the movement of the object 92 make a difference in intensity of stimulus given to the driver's brain. Note that the effect of the stimulus given to the driver's brain can be confirmed as the amount of change detected by the biosensor. It is also possible to update the preference information with the use of the amount of change.

Step S115 is a step in which the conversation data 94a including the driver's reply to the conversation data 93a is detected by the microphone.

In Step S116, the conversation data 94a detected by the wearable device 10 is converted into language data in the conversation data generation unit, enabling the preference information to be updated with the use of the language data. Thus, the classifier in the wearable device 10 can learn what kind of preference information activates the driver's brain by learning the conversation data 93a and the conversation data 94a between the wearable device 10 and the driver, and update the weight coefficient. The conversation data generation unit is capable of learning the movement of the object 92 displayed by the wearable device 10, and the type of voice, the tone of voice, the speed of conversation and the like output in accordance with the movement of the object 92.

FIG. 4 is a block diagram illustrating the wearable device 10, which is a data processing device, and the vehicle. It is preferable that the wearable device 10 and the vehicle be connected with wireless communication or wire communication. A data processing terminal 40, typified by a smartphone or the like, stores object data 41 for displaying the object 92 and classification data 42 of the classifier where the preference information is learned, which enables the object data 41 and the classification data 42 to be portable.

The wearable device 10 includes a control unit 11, a monitoring unit 12, an operation unit 13, an image processing unit 14, an input/output unit 15, and a conversation data generation unit 16. The control unit 11 includes a first memory and a first communication device. The first communication device is capable of communicating with a second communication device and a third communication device, which will be described later.

A vehicle 20 includes a control unit 21, a monitoring unit 22, an operation unit 23, an object generation unit 24, and the like. The control unit 21 includes a second memory and the second communication device. The second communication device can communicate with a satellite 30 or a wireless communication antenna 31. Thus, the second communication device can collect information of the surroundings of the vehicle 20, the traffic information, current invents via the Internet, or the like. Note that the traffic information includes the speed information and location information of a vehicle near the vehicle 20, obtained through the use of the fifth generation mobile communication system (5G) or the like.

The object generation unit 24 is capable of generating objects with the use of the object data 41 of the object 92. Note that the object generation unit 24 may be incorporated in the vehicle 20, or it may be a portable accelerator that can be carried around. The portable accelerator can generate the object data 41 of the object 92 with the use of power of the vehicle by being connected to the vehicle 20. The object data 41 of the object 92 may be generated with the use of the object generation unit prepared in a server computer on the cloud.

The portable accelerator (not illustrated in FIG. 4) includes a GPU (graphics processing unit), a third memory, the third communication device, or the like. The third communication device can be connected to the first communication device and the second communication device via wireless communication. Alternatively, the third communication device can be connected to the second communication device with the use of a hardware interface (for example, USB, Thunderbolt, Ethernet (a registered trademark), eDP (Embedded DisplayPort), OpenLDI (open LVDS display interface), or the like).

The object data 41 of the object 92 is stored in any of a memory included in the data processing terminal 40 typified by a smartphone or the like, the first memory included in the wearable device 10, the third memory included in the portable accelerator, or a memory prepared in a server computer on the cloud, whereby the object data 41 of the object 92 can be developed in the other electronic devices.

The classification data 42 of the classifier with the learned preference information can be stored in any of a memory included in the data processing terminal 40 typified by a smartphone or the like, the first memory included in the wearable device 10, the third memory included in the portable accelerator, or a memory prepared in a server computer on the cloud. Thus, the object data 41 of the object 92 and the classification data 42, as a set, can be developed in the other electronic devices.

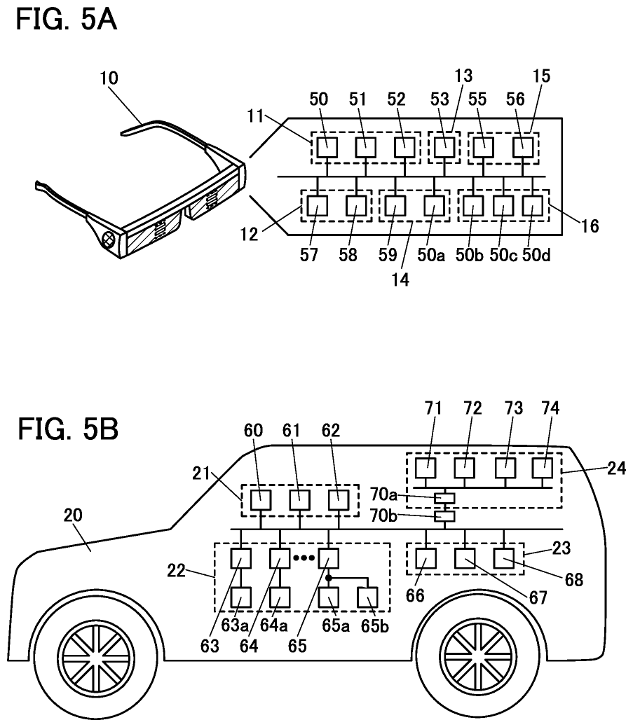

FIG. 5A is a block diagram illustrating the wearable device 10. FIG. 5A is a block diagram describing in more detail the block diagram in FIG. 4.

The wearable device 10 includes the control unit 11, the monitoring unit 12, the operation unit 13, the image processing unit 14, the input/output unit 15, and the conversation data generation unit 16.

The control unit 11 includes a processor 50, a memory 51, a first communication device 52, and the like.

The monitoring unit 12 includes a biosensor 57, an imaging device 58, and the like. The biosensor 57 is capable of detecting body temperature, blood pressure, pulse rate, sweating rate, blood sugar level, red blood cell count, respiratory rate, and the like. As an example, an infrared sensor, a temperature sensor, a humidity sensor, or the like is suitable. It is preferable that the monitoring unit 12 include at least two or more imaging devices 58. A second imaging device is capable of capturing an image of eye surroundings. A first imaging device is capable of capturing an image of a region that can be seen through the wearable device.

The operation unit 13 includes a neural network (CNN) 53 for performing image analysis, or the like.

The image processing unit 14 includes a display device 59 and an image processing device 50a that processes image data to be displayed on the display device 59.

The input/output unit 15 includes a speaker 55 and a microphone 56.

The conversation data generation unit 16 includes a GPU 50b, a memory 50c, and a neural network 50d. The neural network 50d preferably includes a plurality of neural networks. The conversation data generation unit 16 includes a classifier. Note that an algorithm such as a decision tree, support-vector machines, random forests, or a multilayer perceptron may be used as the classifier, for example. An algorithm such as K-means or DBSCAN (densitybased spatial clustering of applications with noise) can be used as the classification model of machine leaning using neural networks.

The conversation data generation unit 16 is capable of conversation generation based on the classification data of the classifier. Neuro-linguistic programming (NLP), deep learning using neural networks, and the like can be used for the conversation generation. As an example, sequence to sequence learning, which is a type of deep learning, is suitable for automatically generating conversation.

FIG. 5B is a block diagram illustrating the vehicle 20. FIG. 5B is a block diagram illustrating the block diagram in FIG. 4 in more detail.

The vehicle 20 includes the control unit 21, the monitoring unit 22, the operation unit 23, the object generation unit 24, and the like.

The control unit 21 includes a processor 60, a memory 61, a second communication device 62, and the like.

The second communication device 62 can communicate with the satellite 30 or the wireless communication antenna 31. The second communication device 62 can collect information of the surroundings of the vehicle 20, the traffic information, current invents that can be searched via the Internet, or the like. Note that, as the traffic information, information such as the speed information and location information of a vehicle near the vehicle 20 can be obtained through the use of the fifth generation mobile communication system (5G).

The monitoring unit 22 includes an engine control unit, and the engine control unit includes a control unit 63 to a control unit 65, a sensor 63a, a sensor 64a, a sensor 65a, and a sensor 65b. It is preferable that the control unit be capable of monitoring one or more sensors. With the control unit monitoring the conditions of the sensors, the engine control unit is capable of control related to driving of the vehicle. For example, the engine control unit is capable of braking control in response to the result from a distance sensor that controls a distance between vehicles.

The operation unit 23 can include a GPU 66, a memory 67, and a neural network 68. The neural network 68 is capable of controlling the engine control unit. It is preferable that the neural network 68 perform inference for driving control by supplying an input layer with output from the sensor included in each of the above-described control units. It is preferable that the neural network 68 have already learned the vehicle control and driving information.

The object generation unit 24 includes a GPU 71, a memory 72, a neural network 73, a third communication device 74, and a connector 70a. The object generation unit 24 can be connected to the control unit 21, the monitoring unit 22, and the operation unit 23 by being connected via the connector 70a to a connector 70b of the vehicle 20. The object generation unit 24 can have a portability when including the connector 70a and the third communication device 74.

Note that the third communication device 74 of the object generation unit 24 can be connected to the second communication device 62 through wireless communication. As another example, the object generation unit 24 may be incorporated in the vehicle 20.

Next, FIG. 6A and FIG. 6B are drawings illustrating configuration examples of a wearable device. In FIG. 6A and FIG. 6B, the wearable device, which is a data processing device, is described as a glasses-like information terminal 900.

FIG. 6A illustrates a perspective view of the glasses-like information terminal 900. The information terminal 900 includes a pair of display devices 901, a pair of housings (a housing 902a and a housing 902b), a pair of optical members 903, a pair of temples 904, and the like.

The information terminal 900 can project an image displayed on the display device 901 onto a display region 906 of the optical member 903. In addition, since the optical members 903 have light-transmitting properties, the user can see images displayed on the display regions 906 that are superimposed on transmission images seen through the optical members 903. Thus, the information terminal 900 is an information terminal capable of AR display or VR display. Note that not only the display device 901 but also the optical members 903 including the display regions 906 and an optical system including a lens 911, a reflective plate 912, and a reflective plane 913 to be described later can be included in the display unit. A micro-LED display can be used as the display device 901. As another example, an organic EL display, an inorganic EL display, a liquid crystal display, or the like can be used as the display device 901. In the case where a liquid crystal display is used as the display device 901, an inorganic light-emitting element can be used as a light source functioning as a backlight.

In addition, a pair of imaging devices 905 capable of taking front images and a pair of imaging device 909 capable of taking images on the user side are provided in the information terminal 900. The imaging devices 905 and the imaging devices 909 are some of the components of an imaging device module. Providing the information terminal 900 with two imaging devices 905 is preferable because an image of an object can be captured 3-dimentionally. Note that the number of imaging devices 905 provided in the information terminal 900 may be one or three or more. The imaging device 905 may be provided in a center portion of a front of the information terminal 900 or may be provided in a front of one or each of the housing 902a and the housing 902b. Furthermore, two imaging devices 905 may be provided in fronts of the housing 902a and the housing 902b.

The imaging devices 909 are capable of detecting the line of sight of the user. Thus, two imaging devices 909 for a right eye and for a left eye are preferably provided. However, in the case where one imaging device can sense the gaze of both eyes, one imaging device 909 may be provided. The imaging devices 909 may be infrared imaging devices capable of detecting infrared rays. The infrared imaging devices are suitable for detecting the iris.

The housing 902a includes a wireless communication device 907 and is capable of supplying a video signal or the like to a housing 902 through the wireless communication device 907. Furthermore, the wireless communication device 907 preferably includes a communication module and communicates with a database. Instead of the wireless communication device 907 or in addition to the wireless communication device 907, a connector that can be connected to a cable 910 for supplying a video signal or a power supply potential may be provided. Furthermore, when the housing 902 is provided with an acceleration sensor, a gyroscope sensor, or the like, the orientation of the user's head can be sensed and an image corresponding to the orientation can also be displayed on the display region 906. Moreover, the housing 902 is preferably provided with the battery, in which case charging can be performed with or without a wire. The battery is preferably incorporated in the pair of temples 904.

The information terminal 900 can include a biosensor. As an example, the information terminal 900 includes a biosensor 921 placed in a position of the temple 904 touching the ear and a biosensor 922 placed in the pad touching the nose. A temperature sensor, an infrared sensor, or the like is preferably used as the biosensor. It is preferable that the biosensor 921 and the biosensor 922 be incorporated in the positions in direct contact with the ear and the nose. The biosensors are capable of detecting the user's biological information. The biological information includes body temperature, blood pressure, pulse rate, sweating rate, blood sugar level, red blood cell count, respiratory rate, and the like. In the case where the biosensor in not in contact with the user, it is preferable that the biological information be detected using the temple area.

The housing 902b is provided with an integrated circuit 908. The integrated circuit 908 includes a control unit, a monitoring unit, an operation unit, an image processing unit, a conversation data generation unit, and the like, although not shown in FIG. 6A. The information terminal 900 also includes the imaging device 905, the wireless communication device 907, the pair of display devices 901, a microphone, a speaker, and the like. It is preferable that the information terminal 900 include a function of generating conversation data, a function of generating images, and the like. The integrated circuit 908 preferably has a function of generating synthetic images for AR display or VR display.

Data communication with an external device can be performed by the wireless communication device 907. For example, when data transmitted from the outside is output to the integrated circuit 908, the integrated circuit 908 can generate image data for AR display or VR display on the basis of the data. Examples of data transmitted from the outside include object data, which is generated in the object generation unit based on an image obtained by the imaging device 905 and transmitted to the object generation unit, the driving information, the topic information, and the like.

Next, a method for projecting an image on the display region 906 of the information terminal 900 will be described with reference to FIG. 6B. The display device 901, the lens 911, and the reflective plate 912 are provided in the housing 902. In addition, the reflective plane 913 functioning as a half mirror is provided in a portion corresponding to the display region 906 of the optical member 903.

Light 915 emitted from the display device 901 passes through the lens 911 and is reflected by the reflective plate 912 to the optical member 903 side. In the optical member 903, the light 915 is fully reflected repeatedly by end surfaces of the optical member 903 and reaches the reflective plane 913, so that an image is projected on the reflective plane 913. Accordingly, the user can see both the light 915 reflected by the reflective plane 913 and transmitted light 916 that has passed through the optical member 903 (including the reflective plane 913).

FIG. 6B illustrates an example in which the reflective plate 912 and the reflective plane 913 each have a curved surface. This can increase optical design flexibility and reduce the thickness of the optical member 903, compared to the case where they have flat surfaces. Note that the reflective plate 912 and the reflective plane 913 may have flat surfaces.

A component having a mirror surface can be used for the reflective plate 912, and the reflective plate 912 preferably has high reflectance. In addition, as the reflective plane 913, a half mirror utilizing reflection of a metal film may be used, but the use of a prism utilizing total reflection or the like can increase the transmittance of the transmitted light 916.

Here, the housing 902 preferably includes a mechanism for adjusting the distance and angle between the lens 911 and the display device 901. This enables focus adjustment, zooming in and out of an image, or the like. One or both of the lens 911 and the display device 901 are configured to be movable in the optical-axis direction, for example.

In addition, the housing 902 preferably includes a mechanism capable of adjusting the angle of the reflective plate 912. The position of the display region 906 where images are displayed can be changed by changing the angle of the reflective plate 912. Thus, the display region 906 can be placed at the most appropriate position in accordance with the position of the user's eye.

The display device of one embodiment of the present invention can be used for the display device 901. Thus, the information terminal 900 can perform display with extremely high resolution.

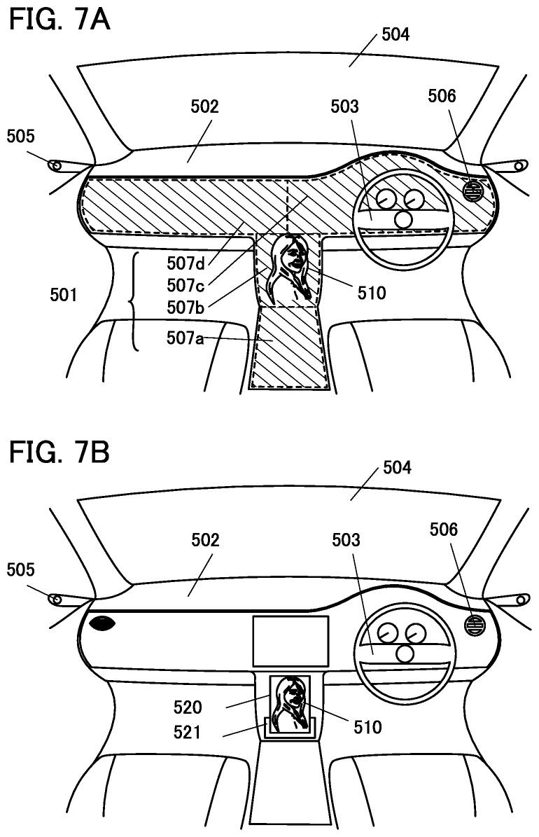

FIG. 7A and FIG. 7B illustrate configuration examples where the object is seen through the data processing device. As an example, the data processing device is incorporated in the vehicle in FIG. 7A. The data processing device is provided with a display unit 501.

Note that although in the example illustrated in FIG. 7A, the display unit 501 is installed in, but not limited to, a right-hand drive vehicle; installation in a left-hand drive vehicle is possible. Here, the vehicle will be described. FIG. 7A illustrates a dashboard 502, a steering wheel 503, a windshield 504, and the like that are arranged around a driver seat and a passenger seat. The display unit 501 is placed in a predetermined position in the dashboard 502, specifically, around the driver, and has a rough T shape. Although one display unit 501 formed of a plurality of display panels (display panels 507a, 507b, 507c, and 507d) is provided along the dashboard 502 in the example illustrated in FIG. 7A, the display unit 501 may be divided and placed in a plurality of places.

Note that the plurality of display panels may have flexibility. In that case, the display unit 501 can be processed into a complicated shape; for example, a structure in which the display unit 501 is provided along a curved surface of the dashboard 502 or the like or a structure in which a display region of the display unit 501 is not provided at a connection portion of the steering wheel, display units of meters, a ventilation duct 506, or the like can easily be achieved.

In addition, a plurality of cameras 505 that take pictures of the situations at the rear side may be provided outside the vehicle. Although the camera 505 is provided instead of a side mirror in the example in FIG. 7A, both the side mirror and the camera may be provided.

As the camera 505, a CCD camera, a CMOS camera, or the like can be used. In addition, an infrared camera may be used in combination with such a camera. The infrared camera, which has a higher output level with a higher temperature of an object, can detect or extract a living body such as a human or an animal.

An object 510 can be displayed on the display unit 501 (the display panels 507a, 507b, 507c, and 507d). It is preferable that the object 510 be displayed on a position that activates the driver's brain. Thus, the position where the object 510 is displayed is not limited to the wearable device 10. The object 510 can be displayed on one or more of the display panels 507a, 507b, 507c, and 507d.

An image captured with the camera 505 can be output to any one or more of the display panels 507a, 507b, 507c, and 507d. The object 510 is capable of generating conversation data with the image as the driving information or topic information. Note that in the case where the data processing device displays the object 510 and outputs conversation data using the image, it is preferable that the image be displayed on the display unit 501 (the display panels 507a, 507b, 507c, and 507d) at the same time. When the data processing device outputs to the driver the conversation data that is related to the image, the driver feels as if conversing with the object 510 and thus the driver's stress can be reduced.

In the case where the display unit 501 displays map information, traffic information, television images, DVD images, or the like, the object 510 is displayed on one or more of the display panels 507a, 507b, 507c, and 507d. When the data processing device outputs to the driver the conversation data that is related to the map information, traffic information, television images, DVD images, or the like, the driver feels as if conversing with the object 510 and thus the driver's stress can be reduced. Note that the number of display panels used in the display unit 501 can be increased depending on the image to be displayed.

An example different from FIG. 7A is shown in FIG. 7B. In FIG. 7B, a cradle 521 for storing a data processing device 520 is provided in the vehicle. The cradle 521 stores the data processing device 520, so that the object 510 is displayed on the display unit included in the data processing device 520. Note that the cradle 521 can connect the data processing device 520 to the vehicle. The data processing device 520 preferably includes the conversation data generation unit, the operation unit, the image processing unit, the display device, the imaging device, the biosensor, the speaker, and the microphone. Note that the cradle 521 preferably has a function of charging the data processing device 520.

As described above, the data processing device of one embodiment of the present invention can facilitate activation of consciousness by conversation or the like. The data processing device is capable of generating conversation data with the use of the driving information, the driver information, the topic information, and the like. The data processing device can also have an extended reality function that associates the conversation data with the movement of the displayed object. The data processing device is capable of generating conversation data with the use of the classifier including the user's preference information. The data processing device is capable of generating conversation data with the use of the biological information detected by the biosensor and the preference information included in the classifier. The data processing device can update the preference information in the classifier with the use of the user's biological information detected by the biosensor and the user's conversation data. Note that the data processing device is a wearable device or an automatic voice response device (AI speaker). The data processing device can be incorporated in a vehicle or an electronic device. Note that, in the case where the data processing device is incorporated in a vehicle or an electronic device without a display device, object display is not performed.

This embodiment can be combined with the description of the other embodiments as appropriate.

### Embodiment 2

This embodiment will show examples of a semiconductor wafer provided with the processor, integrated circuit including a GPU, or the like described in the foregoing embodiment and an electronic component including the integrated circuit. The integrated circuit can also be referred to as a semiconductor device. Thus, the integrated circuit will be described as a semiconductor device in this embodiment.

### <Semiconductor Wafer>

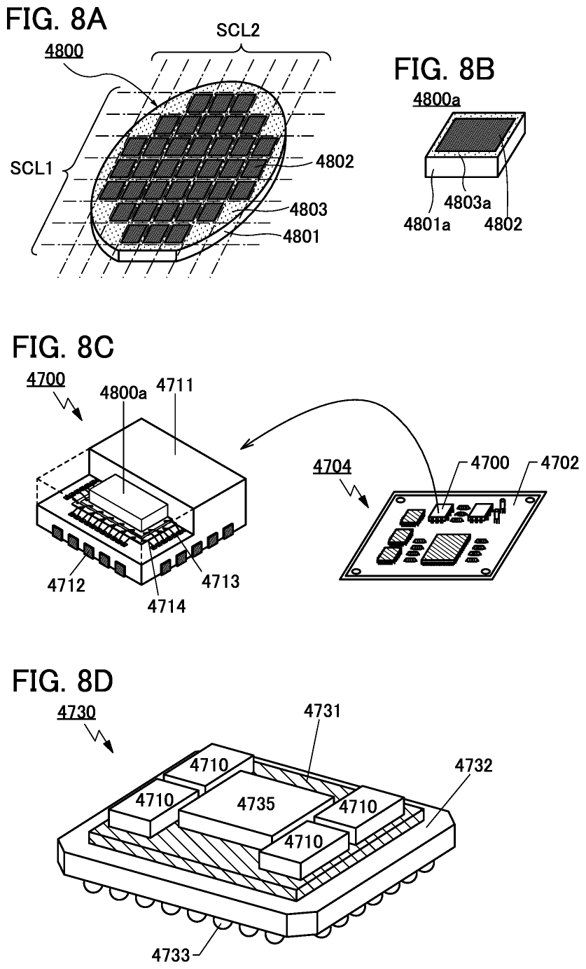

First, an example of a semiconductor wafer provided with a semiconductor device or the like is described with reference to FIG. 8A.

A semiconductor wafer 4800 illustrated in FIG. 8A includes a wafer 4801 and a plurality of circuit portions 4802 provided on the top surface of the wafer 4801. A portion without the circuit portions 4802 on the top surface of the wafer 4801 is a spacing 4803 that is a region for dicing.

The semiconductor wafer 4800 can be formed by forming the plurality of circuit portions 4802 on the surface of the wafer 4801 by a pre-process. After that, a surface of the wafer 4801 opposite to the surface provided with the plurality of circuit portions 4802 may be ground to thin the wafer 4801. Through this step, warpage or the like of the wafer 4801 is reduced and the size of the component can be reduced.

Next, a dicing step is performed. The dicing is carried out along scribe lines SCL1 and scribe lines SCL2 (sometimes referred to as dicing lines or cutting lines) indicated by dashed-dotted lines. To perform the dicing step easily, the spacing 4803 is preferably arranged such that a plurality of scribe lines SCL1 are parallel to each other, a plurality of scribe lines SCL2 are parallel to each other, and the scribe lines SCL1 and the scribe lines SCL2 intersect each other perpendicularly. Note that the scribe lines are preferably set such that the number of chips to be obtained is maximized.

With the dicing step, a chip 4800a illustrated in FIG. 8B can be cut out from the semiconductor wafer 4800. The chip 4800a includes a wafer 4801a, the circuit portion 4802, and a spacing 4803a. Note that it is preferable to make the spacing 4803a as small as possible. Here, it is preferred that the width of the spacing 4803 between adjacent circuit portions 4802 be substantially the same as the width of the scribe line SCL1 or the scribe line SCL2.

The shape of the element substrate of one embodiment of the present invention is not limited to the shape of the semiconductor wafer 4800 illustrated in FIG. 8A. The element substrate may be a rectangular semiconductor wafer, for example. The shape of the element substrate can be changed as appropriate, depending on a process for fabricating an element and an apparatus for fabricating the element.

### <Electronic Component>

FIG. 8C is a perspective view of an electronic component 4700 and a substrate (a circuit substrate 4704) on which the electronic component 4700 is mounted. The electronic component 4700 in FIG. 8C includes the chip 4800a in a mold 4711. As the chip 4800a, the memory device of one embodiment of the present invention can be used, for example.

To illustrate the inside of the electronic component 4700, some portions are omitted in FIG. 8C. The electronic component 4700 includes a land 4712 outside the mold 4711. The land 4712 is electrically connected to an electrode pad 4713, and the electrode pad 4713 is electrically connected to the chip 4800a via a wire 4714. The electronic component 4700 is mounted on a printed circuit board 4702, for example. A plurality of such electronic components are combined and electrically connected to each other on the printed circuit board 4702; thus, the circuit substrate 4704 is completed.

FIG. 8D is a perspective view of an electronic component 4730. The electronic component 4730 is an example of an SiP (System in package) or an MCM (Multi Chip Module). In the electronic component 4730, an interposer 4731 is provided over a package substrate 4732 (a printed circuit board), and a semiconductor device 4735 and a plurality of semiconductor devices 4710 are provided over the interposer 4731.

Examples of the semiconductor devices 4710 include the chip 4800a, the semiconductor device described in the foregoing embodiment, and a high bandwidth memory (HBM). Moreover, an integrated circuit (a semiconductor device) such as a CPU, a GPU, an FPGA, or a memory device can be used as the semiconductor device 4735.

As the package substrate 4732, a ceramic substrate, a plastic substrate, a glass epoxy substrate, or the like can be used. As the interposer 4731, a silicon interposer, a resin interposer, or the like can be used.

The interposer 4731 includes a plurality of wirings and has a function of electrically connecting a plurality of integrated circuits with different terminal pitches. The plurality of wirings have a single-layer structure or a multi-layer structure. The interposer 4731 has a function of electrically connecting an integrated circuit provided on the interposer 4731 to an electrode provided on the package substrate 4732. Accordingly, the interposer is sometimes referred to as a redistribution substrate or an intermediate substrate. A through electrode may be provided in the interposer 4731 and used to electrically connect the integrated circuit and the package substrate 4732. In the case of using a silicon interposer, a TSV (Through Silicon Via) can also be used as the through electrode.

A silicon interposer is preferably used as the interposer 4731. The silicon interposer can be manufactured at lower cost than an integrated circuit because the silicon interposer need not to be provided with an active element. Moreover, since wirings of the silicon interposer can be formed through a semiconductor process, the formation of minute wirings, which is difficult for a resin interposer, is easily achieved.

An HBM needs to be connected to many wirings to achieve a wide memory bandwidth. Therefore, minute wirings are required to be formed densely on an interposer on which an HBM is mounted. For this reason, a silicon interposer is preferably used as the interposer on which an HBM is mounted.

In an SiP, an MCM, and the like using a silicon interposer, a decrease in reliability due to a difference in expansion coefficient between an integrated circuit and the interposer is less likely to occur. Furthermore, the surface of a silicon interposer has high planarity, so that a poor connection between the silicon interposer and an integrated circuit provided thereon is less likely to occur. It is particularly preferable to use a silicon interposer for a 2.5D package (2.5D mounting) in which a plurality of integrated circuits are arranged side by side on an interposer.

A heat sink (radiator plate) may be provided to overlap with the electronic component 4730. When a heat sink is provided, the heights of integrated circuits provided on the interposer 4731 are preferably the same. For example, in the electronic component 4730 described in this embodiment, the heights of the semiconductor devices 4710 and the semiconductor device 4735 are preferably the same.

An electrode 4733 may be provided on the bottom of the package substrate 4732 to mount the electronic component 4730 on another substrate. FIG. 8D illustrates an example in which the electrode 4733 is formed of a solder ball. Solder balls are provided in a matrix on the bottom of the package substrate 4732, whereby a BGA (Ball Grid Array) can be achieved. Alternatively, the electrode 4733 may be formed of a conductive pin. When conductive pins are provided in a matrix on the bottom of the package substrate 4732, a PGA (Pin Grid Array) can be achieved.

The electronic component 4730 can be mounted on another substrate in a variety of manners other than a BGA and a PGA. For example, an SPGA (Staggered Pin Grid Array), an LGA (Land Grid Array), a QFP (Quad Flat Package), a QFJ (Quad Flat J-leaded package), or a QFN (Quad Flat Non-leaded package) can be employed.

Note that this embodiment can be combined with any of the other embodiments in this specification as appropriate.

### Embodiment 3

This embodiment will describe an example of an arithmetic processing device that can include the semiconductor device, such as the memory device described in any of the above embodiments.

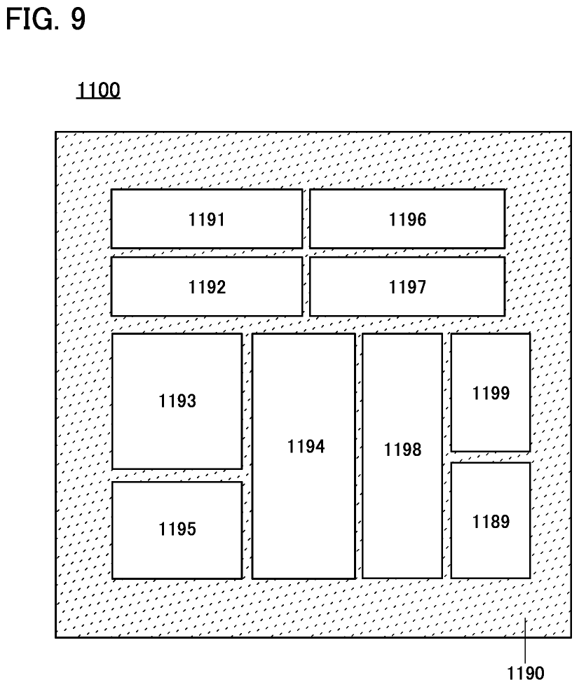

FIG. 9 is a block diagram of a central processing unit 1100. FIG. 9 illustrates a configuration example of a CPU applicable to the central processing unit 1100.

The central processing unit 1100 illustrated in FIG. 9 includes, over a substrate 1190, an ALU 1191 (Arithmetic logic unit), an ALU controller 1192, an instruction decoder 1193, an interrupt controller 1194, a timing controller 1195, a register 1196, a register controller 1197, a bus interface 1198, a cache 1199, and a cache interface 1189. A semiconductor substrate, an SOI substrate, a glass substrate, or the like is used as the substrate 1190. The central processing unit 1100 may also include a rewritable ROM and a ROM interface. The cache 1199 and the cache interface 1189 may be provided in a separate chip.

The cache 1199 is connected via the cache interface 1189 to a main memory provided in another chip. The cache interface 1189 has a function of supplying part of data held in the main memory to the cache 1199. The cache 1199 has a function of retaining the data.

The central processing unit 1100 illustrated in FIG. 9 is only an example with a simplified configuration, and the actual central processing unit 1100 has a variety of configurations depending on the application. For example, the central processing unit may have a GPU-like configuration in which a plurality of cores each including the central processing unit 1100 in FIG. 9 or an arithmetic circuit operate in parallel. The number of bits that the central processing unit 1100 can handle with an internal arithmetic circuit or a data bus can be 1, 8, 16, 32, or 64, for example. When the number of bits that the data bus can handle is 1, it is preferable that three values “1”, “0”, “−1” be handled.

An instruction input to the central processing unit 1100 through the bus interface 1198 is input to the instruction decoder 1193 and decoded, and then input to the ALU controller 1192, the interrupt controller 1194, the register controller 1197, and the timing controller 1195.

The ALU controller 1192, the interrupt controller 1194, the register controller 1197, and the timing controller 1195 conduct various controls in accordance with the decoded instruction. Specifically, the ALU controller 1192 generates signals for controlling the operation of the ALU 1191. The interrupt controller 1194 judges and processes an interrupt request from an external input/output device or a peripheral circuit on the basis of its priority and a mask state while the central processing unit 1100 is executing a program. The register controller 1197 generates the address of the register 1196, and reads/writes data from/to the register 1196 in accordance with the state of the central processing unit 1100.

The timing controller 1195 generates signals for controlling operation timings of the ALU 1191, the ALU controller 1192, the instruction decoder 1193, the interrupt controller 1194, and the register controller 1197. For example, the timing controller 1195 includes an internal clock generator for generating an internal clock signal on the basis of a reference clock signal, and supplies the internal clock signal to the above circuits.

In the central processing unit 1100 in FIG. 9, a memory device is provided in the register 1196 and the cache 1199.

In the central processing unit 1100 in FIG. 9, the register controller 1197 selects operation of retaining data in the register 1196 in accordance with an instruction from the ALU 1191. That is, the register controller 1197 selects whether data is held by a flip-flop or by a capacitor in a memory cell included in the register 1196. When data retention by the flip-flop is selected, power supply voltage is supplied to the memory cell in the register 1196. When data retention by the capacitor is selected, the data is rewritten into the capacitor, and supply of power supply voltage to the memory cell in the register 1196 can be stopped.

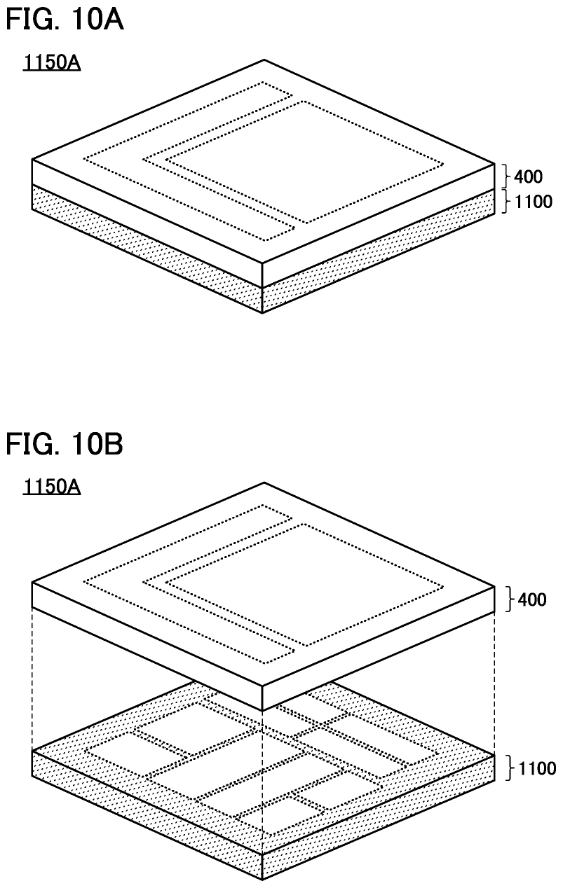

The semiconductor device described in the above embodiment and the central processing unit 1100 can be provided to overlap each other. FIG. 10A and FIG. 10B are perspective views of a semiconductor device 1150A. The semiconductor device 1150A includes the semiconductor device 400 functioning as a memory device over the central processing unit 1100. The central processing unit 1100 and the semiconductor device 400 have an overlap region. For easy understanding of the structure of the semiconductor device 1150A, the central processing unit 1100 and the semiconductor device 400 are separated from each other in FIG. 10B.

Overlapping the semiconductor device 400 and the central processing unit 1100 can shorten the physical distance therebetween. Accordingly, the communication speed therebetween can be increased. Moreover, a short physical distance leads to lower power consumption.

When an OS NAND memory device is used as the semiconductor device 400, some or all of the memory cells included in the semiconductor device 400 can function as RAM. Thus, the semiconductor device 400 can function as a main memory. The semiconductor device 400 functioning as the main memory is connected to the cache 1199 through the cache interface 1189.

Whether the semiconductor device 400 functions as the main memory (RAM) or storage is determined in accordance with a signal supplied from the central processing unit 1100. Thus, the central processing unit 1100 can make some of the memory cells in the semiconductor device 400 function as RAM in accordance with a signal the central processing unit 1100 supplies.

In the semiconductor device 400, some of the memory cells can function as the RAM and the other memory cells as the storage. When an OS NAND memory device is used as the semiconductor device 400, the semiconductor device 400 has both the function of the main memory and the function of the storage. The semiconductor device 400 of one embodiment of the present invention can function as a universal memory, for example.

In the case where the semiconductor device 400 is used as the main memory, the memory capacity can be increased or decreased as needed. In the case where the semiconductor device 400 is used as a cache, the memory capacity can be increased or decreased as needed.

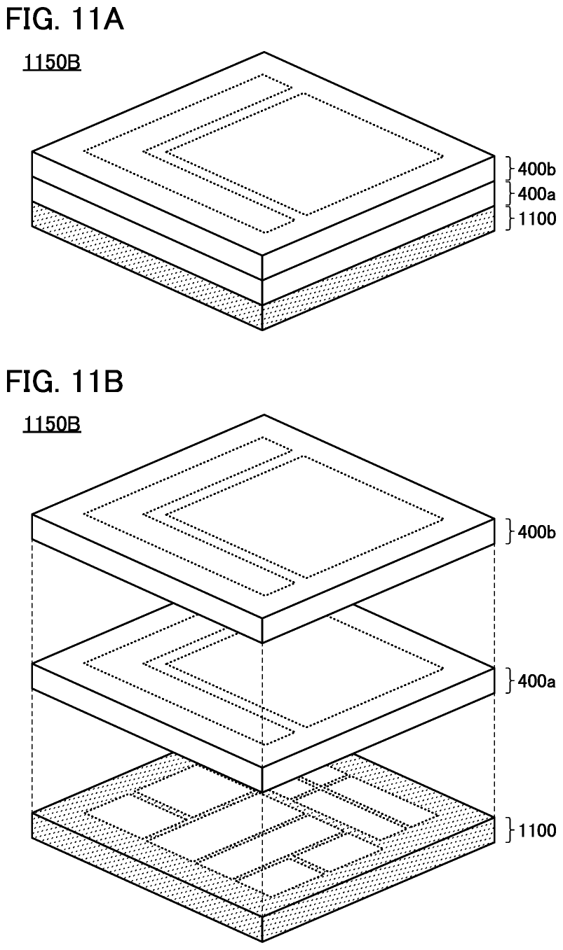

A plurality of semiconductor devices 400 may be provided to overlap the central processing unit 1100. FIG. 11A and FIG. 11B are perspective views of a semiconductor device 1150B. The semiconductor device 1150B includes a semiconductor device 400a and a semiconductor device 400b over the central processing unit 1100. The central processing unit 1100, the semiconductor device 400a, and the semiconductor device 400b have an overlap region. For easy understanding of the structure of the semiconductor device 1150B, the central processing unit 1100, the semiconductor device 400a, and the semiconductor device 400b are separated from each other in FIG. 11B.

The semiconductor devices 400a and 400b function as memory devices. For example, a NOR memory device may be used as the semiconductor device 400a. A NAND memory device may be used as the semiconductor device 400b. A NOR memory device can operate at higher speed than a NAND memory device; hence, for example, part of the semiconductor device 400a can be used as the main memory and/or the cache 1199. Note that the stacking order of the semiconductor device 400a and the semiconductor device 400b may be reverse.

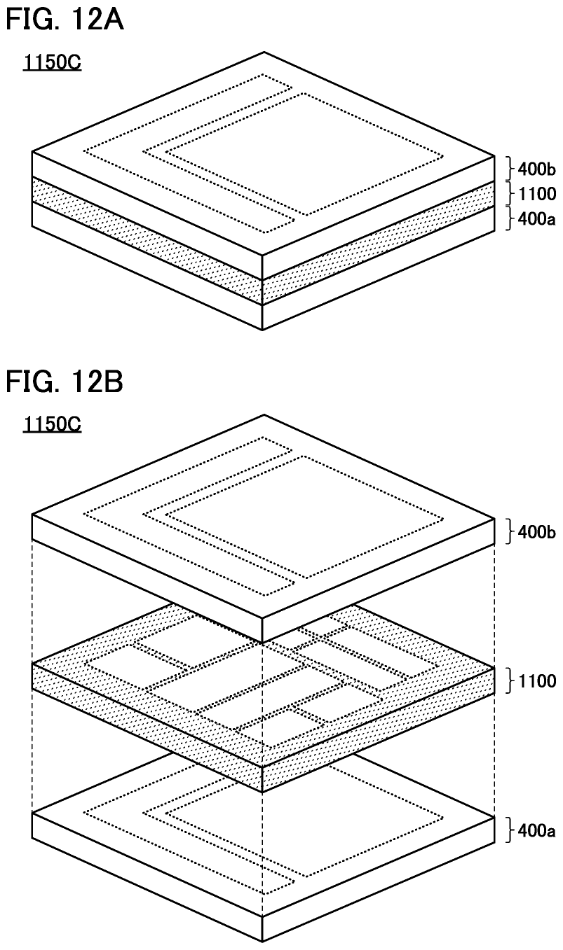

FIG. 12A and FIG. 12B are perspective views of a semiconductor device 1150C. In the semiconductor device 1150C, the central processing unit 1100 is provided between the semiconductor device 400a and the semiconductor device 400b. Thus, the central processing unit 1100, the semiconductor device 400a, and the semiconductor device 400b have an overlap region. For easy understanding of the structure of the semiconductor device 1150C, the central processing unit 1100, the semiconductor device 400a, and the semiconductor device 400b are separated from each other in FIG. 12B.

With the structure of the semiconductor device 1150C, the communication speed between the semiconductor device 400a and the central processing unit 1100 and the communication speed between the semiconductor device 400b and the central processing unit 1100 can be both increased. Moreover, power consumption can be reduced, compared to the semiconductor device 1150B.

Note that this embodiment can be combined with any of the other embodiments in this specification as appropriate.

### Embodiment 4

In this embodiment, application examples of the memory device of one embodiment of the present invention will be described.

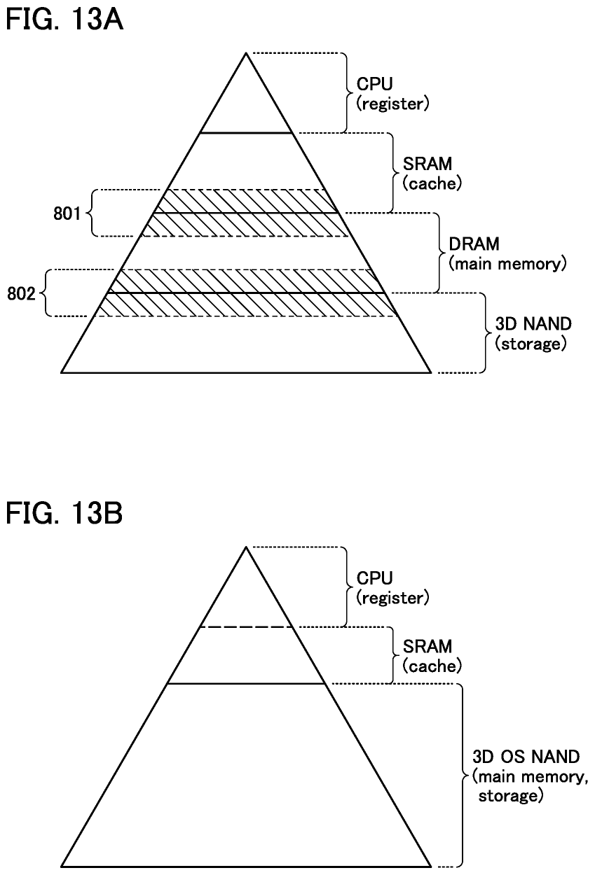

In general, a variety of memory devices are used in semiconductor devices such as computers in accordance with the intended use. FIG. 13A illustrates the hierarchy of various memory devices used in a semiconductor device. The memory devices at the upper levels require a higher operating speed, whereas the memory devices at the lower levels require a larger memory capacity and a higher memory density. FIG. 13A shows, sequentially from the top level, a memory included as a register in an arithmetic processing device such as a CPU, a static random access memory (SRAM), a dynamic random access memory (DRAM), and a 3D NAND memory.

A memory included as a register in an arithmetic processing device such as a CPU is used for temporary storage of arithmetic operation results, for example, and thus is very frequently accessed by the arithmetic processing device. Accordingly, rapid operation is more important than the memory capacity of the memory. The register also has a function of retaining settings of the arithmetic processing device, for example.

An SRAM is used for a cache, for example. The cache has a function of duplicating and retaining part of data held in a main memory. Duplicating frequently used data and holding the duplicated data in the cache facilitates rapid data access. The cache requires a smaller memory capacity than the main memory but a higher operating speed than the main memory. Data that is rewritten in the cache is duplicated, and the duplicated data is supplied to the main memory.

A DRAM is used for the main memory, for example. The main memory has a function of holding a program and data that are read from the storage. The memory density of a DRAM is approximately 0.1 to 0.3 Gbit/mm².

A 3D NAND memory is used for the storage, for example. The storage has a function of holding data that needs to be stored for a long time and programs used in an arithmetic processing device, for example. Therefore, the storage needs to have a high memory capacity and a high memory density rather than operating speed. The memory density of the memory device used as the storage is approximately 0.6 to 6.0 Gbit/mm².

The memory device of one embodiment of the present invention operates fast and can hold data for a long time. The memory device of one embodiment of the present invention can be favorably used as a memory device in a boundary region 801 that includes both the level including the cache and the level including the main memory. The memory device of one embodiment of the present invention can be favorably used as a memory device in a boundary region 802 that includes both the level including the main memory and the level including the storage.

The memory device of one embodiment of the present invention can be favorably used at both the level including the main memory and the level including the storage. The memory device of one embodiment of the present invention can be favorably used at the level including the cache. FIG. 13B illustrates the hierarchy of various memory devices different from that in FIG. 13A.

FIG. 13B shows, sequentially from the top level, a memory included as a register in an arithmetic processing device such as a CPU, an SRAM used as a cache, and a 3D OS NAND memory. The memory device of one embodiment of the present invention can be used for the cache, main memory, and storage. When a high-speed memory of 1 GHz or higher is required as the cache, the cache is included in an arithmetic processing device such as a CPU.

The memory device of one embodiment of the present invention is not limited to a NAND type, and may alternatively be a NOR type or a combination of a NAND type and a NOR type.

The memory device of one embodiment of the present invention can be used, for example, as memory devices of a variety of electronic devices (e.g., information terminals, computers, smartphones, e-book readers, digital still cameras, video cameras, video recording/reproducing devices, navigation systems, and game machines). The memory device of one embodiment of the present invention can also be used for image sensors, IoT (Internet of Things), healthcare, and the like. Here, the computers refer not only to tablet computers, notebook computers, and desktop computers, but also to large computers such as server systems.

Furthermore, the data processing device of one embodiment of the present invention can be used, for example, as data processing devices of a variety of electronic devices (e.g., information terminals, computers, smartphones, e-book readers, digital still cameras, video cameras, video recording/reproducing devices, navigation systems, and game machines). The data processing device of one embodiment of the present invention can also be used for image sensors, IoT (Internet of Things), healthcare, and the like. Here, the computers refer not only to tablet computers, notebook computers, and desktop computers, but also to large computers such as server systems.

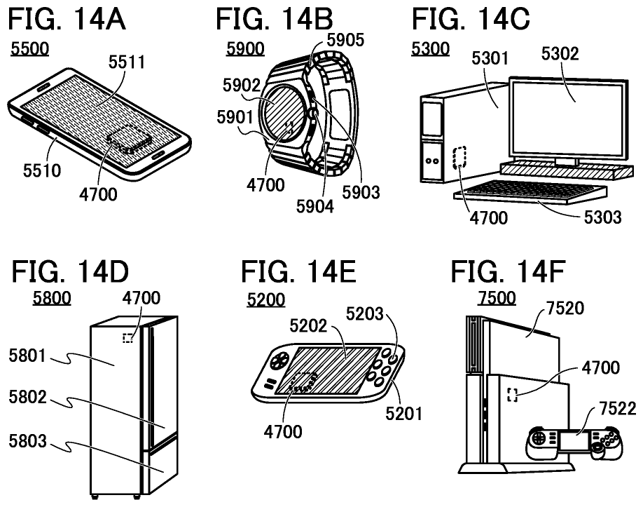

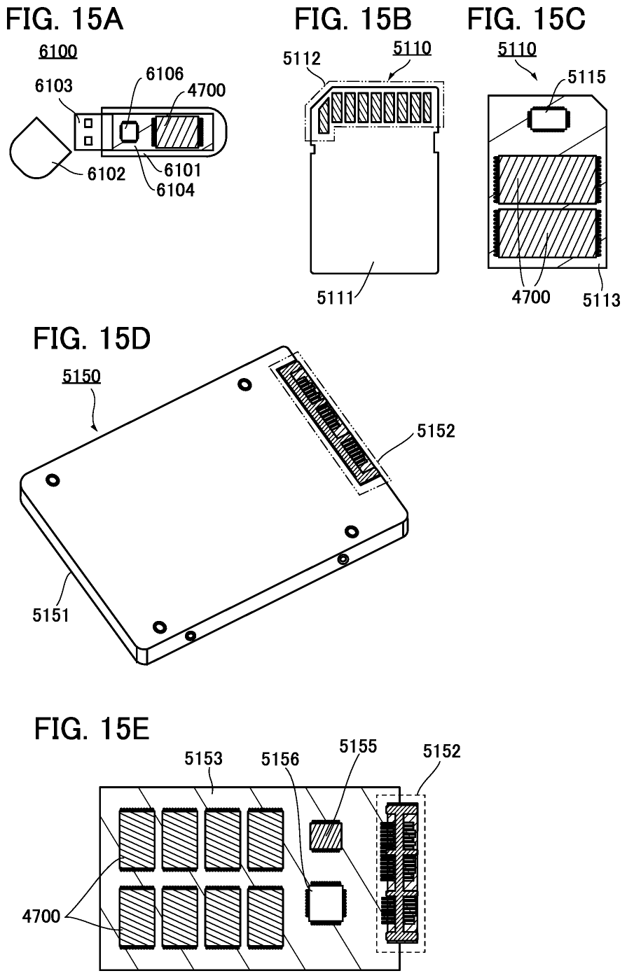

Examples of electronic device including the data processing device and the memory device of one embodiment of the present invention will be described. FIG. 14A to FIG. 14F and FIG. 15A to 15E show that the electronic component 4700 or the electronic component 4730, which includes the data processing device and the memory device, is included in an electronic device.

### [Mobile Phone]

An information terminal 5500 illustrated in FIG. 14A is a mobile phone (a smartphone), which is a type of information terminal. The information terminal 5500 includes a housing 5510 and a display unit 5511. As input interfaces, a touch panel and a button are provided in the display unit 5511 and the housing 5510, respectively. An object can be displayed on the display unit 5511. The information terminal 5500 preferably includes a conversation data generation unit, a speaker, and a microphone.

By using the memory device of one embodiment of the present invention, the information terminal 5500 can hold a temporary file generated at the time of executing an application (e.g., a web browser's cache).

### [Wearable Terminal]

FIG. 14B illustrates an information terminal 5900 as an example of a wearable terminal. The information terminal 5900 includes a housing 5901, a display unit 5902, an operation switch 5903, an operation switch 5904, a band 5905, and the like. The information terminal 5900 preferably includes a biosensor. When the information terminal 5900 includes the biosensor, the biological information of the user, such as steps, body temperature, blood pressure, pulse rate, sweating rate, blood sugar level, and respiratory rate can be detected. The biological information can serve as preference information related to the user's movement and enables a classifier to be updated.

Like the information terminal 5500, the wearable terminal can hold a temporary file generated at the time of executing an application, by using the memory device of one embodiment of the present invention.

### [Information Terminal]

FIG. 14C illustrates a desktop information terminal 5300. The desktop information terminal 5300 includes a main body 5301 of the information terminal, a display unit 5302, and a keyboard 5303. The main body 5301 is capable of updating the classifier with the history information such as Internet browsing history and video watching history, serving as the preference information related to the field of the user's interest.

Like the information terminal 5500, the desktop information terminal 5300 can hold a temporary file generated at the time of executing an application, by using the memory device of one embodiment of the present invention.

Note that although FIGS. 14A to 14C illustrate a smartphone, a wearable terminal, and a desktop information terminal as examples of electronic device, one embodiment of the present invention can also be applied to an information terminal other than a smartphone, a wearable terminal, and a desktop information terminal. Examples of information terminals other than a smartphone, a wearable terminal, and a desktop information terminal include a PDA (Personal Digital Assistant), a laptop information terminal, and a workstation.

### [Consumer Electronics]

FIG. 14D illustrates an electric refrigerator-freezer 5800 as an example of consumer electronics. The electric refrigerator-freezer 5800 includes a housing 5801, a refrigerator door 5802, a freezer door 5803, and the like. For example, the electric refrigerator-freezer 5800 is compatible with the IoT (Internet of Things). The electric refrigerator-freezer 5800 is capable of updating the classifier with the history information such as the storing history of the contents stored in the refrigerator, serving as the preference information related to the diet and health of the user.

The memory device of one embodiment of the present invention can be used in the electric refrigerator-freezer 5800. The electric refrigerator-freezer 5800 can transmit and receive data on food stored in the electric refrigerator-freezer 5800 and food expiration dates, for example, to/from an information terminal and the like via the Internet. In the electric refrigerator-freezer 5800, the memory device can hold a temporary file generated at the time of transmitting the data.

Here, an electric refrigerator-freezer is described as an example of a household appliance; other examples of household appliances include a vacuum, a microwave oven, an electric oven, a rice cooker, a water heater, an IH cooker, a water server, a heating-cooling combination appliance such as an air conditioner, a washing machine, a drying machine, and an audio visual appliance.

### [Game Machines]

FIG. 14E illustrates a portable game machine 5200 as an example of a game machine. The portable game machine 5200 includes a housing 5201, a display unit 5202, a button 5203, and the like.

FIG. 14F illustrates a stationary game machine 7500 as another example of a game machine. The stationary game machine 7500 includes a main body 7520 and a controller 7522. The controller 7522 can be connected to the main body 7520 with or without a wire. Although not illustrated in FIG. 14F, the controller 7522 can include a display unit that displays a game image, and an input interface besides a button, such as a touch panel, a stick, a rotating knob, and a sliding knob, for example. The shape of the controller 7522 is not limited to that in FIG. 14F and may be changed variously in accordance with the genres of games. For example, in a shooting game such as an FPS (First Person Shooter), a gun-shaped controller having a trigger button can be used. As another example, in a music game or the like, a controller having a shape of a music instrument, audio equipment, or the like can be used. Furthermore, the stationary game machine may include a camera, a depth sensor, a microphone, and the like so that the game player can play a game using a gesture and/or a voice instead of a controller.

Videos displayed on the game machine can be output with a display device such as a television device, a personal computer display, a game display, and a head-mounted display. Note that the game machine is capable of updating the classifier with the history information such as the types of games played by the user or usage history such as the playing time, serving as the preference information of the field of the user's interest.

By using the memory device described in the above embodiment in the portable game machine 5200 and the stationary game machine 7500, low power consumption can be achieved in the portable game machine 5200 and the stationary game machine 7500. Moreover, heat generation from a circuit can be reduced owing to low power consumption; thus, the influence of heat generation on the circuit, the peripheral circuit, and the module can be reduced.

Moreover, with the use of the memory device described in the above embodiment, the portable game machine 5200 and the stationary game machine 7500 can hold a temporary file necessary for arithmetic operation that occurs during game play.

As examples of game machines, FIG. 14E illustrates a portable game machine and FIG. 14F illustrates a home-use stationary game machine; however, the electronic device of one embodiment of the present invention is not limited thereto. Other examples of the electronic device of one embodiment of the present invention include an arcade game machine installed in an entertainment facility (e.g., a game center and an amusement park) and a throwing machine for batting practice, installed in sports facilities.

### [Expansion Device for PC]

The memory device described in the foregoing embodiment can be used in a portable accelerator for a vehicle, a PC (personal computer), or other electronic devices, and an expansion device for an information terminal.

FIG. 15A illustrates, as an example of the expansion device, a portable expansion device 6100 that is externally attached to a vehicle, a PC, or other electronic devices and includes a chip capable of storing data. The object data for displaying the object and the classification data of the classifier, which are described in the above embodiment, can be stored in the expansion device 6100. When the expansion device 6100 is connected to a PC with a universal serial bus (USB), for example, data can be stored in the chip. FIG. 15A illustrates the portable expansion device 6100; however, the expansion device of one embodiment of the present invention is not limited to this and may be a relatively large expansion device including a cooling fan or the like, for example.

The expansion device 6100 includes a housing 6101, a cap 6102, a USB connector 6103, and a substrate 6104. The substrate 6104 is held in the housing 6101. The substrate 6104 is provided with a circuit for driving the memory device or the like described in the foregoing embodiment. For example, the substrate 6104 is provided with the electronic component 4700 and a controller chip 6106. The USB connector 6103 functions as an interface for connection to an external device.

### [SD Card]

The memory device described in the above embodiment can be used in an SD card that can be attached to electronic devices such as an information terminal and a digital camera. The object data for displaying the object and the classification data of the classifier, which are described in the above embodiment, can be stored in the SD card.

FIG. 15B is a schematic external diagram of an SD card, and FIG. 15C is a schematic diagram illustrating the internal structure of the SD card. An SD card 5110 includes a housing 5111, a connector 5112, and a substrate 5113. The connector 5112 functions as an interface for connection to an external device. The substrate 5113 is held in the housing 5111. The substrate 5113 is provided with a memory device and a circuit for driving the memory device. For example, the substrate 5113 is provided with the electronic component 4700 and a controller chip 5115. Note that the circuit configurations of the electronic component 4700 and the controller chip 5115 are not limited to those described above and can be changed as appropriate depending on circumstances. For example, a write circuit, a row driver, a read circuit, and the like that are provided in an electronic component may be incorporated into the controller chip 5115 instead of the electronic component 4700.

When the electronic component 4700 is also provided on the back side of the substrate 5113, the capacity of the SD card 5110 can be increased. In addition, a wireless chip with a radio communication function may be provided on the substrate 5113. This enables wireless communication between an external device and the SD card 5110, making it possible to write/read data to/from the electronic component 4700.

### [SSD]

The memory device described in the above embodiment can be used in a solid state drive (SSD) that can be attached to electronic devices such as information terminals. The object data for displaying the object and the classification data of the classifier, which are described in the above embodiment, can be stored in the SSD.

FIG. 15D is a schematic external diagram of an SSD, and FIG. 15E is a schematic diagram of the internal structure of the SSD. An SSD 5150 includes a housing 5151, a connector 5152, and a substrate 5153. The connector 5152 functions as an interface for connection to an external device. The substrate 5153 is held in the housing 5151. The substrate 5153 is provided with a memory device and a circuit for driving the memory device. For example, the substrate 5153 is provided with the electronic component 4700, a memory chip 5155, and a controller chip 5156. When the electronic component 4700 is also provided on the back side of the substrate 5153, the capacity of the SSD 5150 can be increased. A work memory is incorporated into the memory chip 5155. For example, a DRAM chip can be used as the memory chip 5155. A processor, an ECC circuit, and the like are incorporated into the controller chip 5156. Note that the circuit configurations of the electronic component 4700, the memory chip 5155, and the controller chip 5115 are not limited to those described above and can be changed as appropriate depending on circumstances. For example, a memory functioning as a work memory may also be provided in the controller chip 5156.

The hardware in the data processing device includes a first arithmetic processing device, a second arithmetic processing device, a first memory device, and the like. The second arithmetic processing device includes a second memory device.

As the first arithmetic processing device, a central processing unit such as an Noff OS CPU is preferably used, for example. The Noff OS CPU includes a memory unit using OS transistors (e.g., a nonvolatile memory), and has a function of storing necessary data into the memory unit and stopping power supply to the CPU when it does not need to operate. The use of the Noff OS CPU as the first arithmetic processing device can reduce the power consumption of the data processing device.

As the second arithmetic processing device, a GPU or an FPGA can be used, for example. Note that as the second arithmetic processing device, an AI OS accelerator is preferably used. The AI OS accelerator is composed of OS transistors and includes an arithmetic unit such as a product-sum operation circuit. The power consumption of the AI OS accelerator is lower than that of a common GPU and the like. The use of the AI OS accelerator as the second arithmetic processing device can reduce the power consumption of the data processing device.

As the first memory device and the second memory device, the memory device of one embodiment of the present invention is preferably used. For example, the 3D OS NAND memory device is preferably used. The 3D OS NAND memory device can function as a cache, a main memory, and storage. The use of the 3D OS NAND memory device facilitates fabrication of a non-von Neumann computer system.

The power consumption of the 3D OS NAND memory device is lower than that of a 3D NAND memory device using Si transistors. The use of the 3D OS NAND memory device as the memory devices can reduce the power consumption of the data processing device. In addition, the 3D OS NAND memory device can function as a universal memory, thereby reducing the number of components included in the data processing device.

When the semiconductor device constituting the hardware is configured with the semiconductor device including OS transistors, the hardware including the central processing unit, the arithmetic processing device, and the memory device can be easily monolithic. Making the hardware monolithic facilitates a further reduction in power consumption as well as a reduction in size, weight, and thickness.

Note that the configurations described in this embodiment can be used in combination with the description in any of the other embodiments as appropriate.

### REFERENCE NUMERALS

:SCL1: scribe line, SCL2: scribe line, 10: wearable device, 11: control unit, 12: monitoring unit, 13: operation unit, 14: image processing unit, 15: input/output unit, 16: conversation data generation unit, 20: vehicle, 21: control unit, 22: monitoring unit, 23: operation unit, 24: object generation unit, 30: satellite, 31: wireless communication antenna, 40: data processing terminal, 41: object data, 42: classification data, 50: processor, 50a: image processing device, 50b: GPU, 50c: memory, 50d: neural network, 51: memory, 52: communication device, 55: speaker, 56: microphone, 57: biosensor, 58: imaging device, 59: display device, 60: processor, 61: memory, 62: communication device, 63: control unit, 63a: sensor, 64a: sensor, 65: control unit, 65a: sensor, 65b: sensor, 66: GPU, 67: memory, 68: neural network, 70a: connector, 70b: connector, 71: GPU, 72: memory, 73: neural network, 74: communication device, 80: automatic voice response device, 81: speaker, 82: microphone, 83: biosensor, 84: housing, 91: object, 92: object, 93: conversation data, 93a: conversation data, 94: conversation data, 94a: conversation data, 400: semiconductor device, 400a: semiconductor device, 400b: semiconductor device, 501: display unit, 502: dashboard, 503: steering wheel, 504: windshield, 505: camera, 506: ventilation duct, 507a: display panel, 507b: display panel, 507c: display panel, 507d: display panel, 510: object, 520: data processing device, 521: cradle, 801: boundary region, 802: boundary region, 900: information terminal, 901: display device, 902: housing, 902a: housing, 902b: housing, 903: optical member, 904: temple, 905: imaging device, 906: display region, 907: wireless communication device, 908: integrated circuit, 909: imaging device, 910: cable, 911: lens, 912: reflective plate, 913: reflective plate, 915: light, 916: transmitted light, 921: biosensor, 922: biosensor, 1100: central processing unit, 1150A: semiconductor device, 1150B: semiconductor device, 1150C: semiconductor device, 1189: cache interface, 1190: substrate, 1191: ALU, 1192: ALU controller, 1193: instruction decoder, 1194: interrupt controller, 1195: timing controller, 1196: register, 1197: register controller, 1198: bus interface, 1199: cache, 4700: electronic component, 4702: printed circuit board, 4704: circuit substrate, 4710: semiconductor device, 4711: mold, 4712: land, 4713: electrode pad, 4714: wire, 4730: electronic component, 4731: interposer, 4732: package substrate, 4733: electrode, 4735: semiconductor device, 4800: semiconductor wafer, 4800a: chip, 4801: wafer, 4801a: wafer, 4802: circuit portion, 4803: spacing, 4803a: spacing, 5110: SD card, 5111: housing, 5112: connector, 5113: substrate, 5115: controller chip, 5150: SSD, 5151: housing, 5152: connector, 5153: substrate, 5155: memory chip, 5156: controller chip, 5200: portable game machine, 5201: housing, 5202: display unit, 5203: button, 5300: desktop information terminal, 5301: main body, 5302: display unit, 5303: keyboard, 5500: information terminal, 5510: housing, 5511: display unit, 5800: electric refrigerator-freezer, 5801: housing, 5802: refrigerator door, 5803: freezer door, 5900: information terminal, 5901: housing, 5902: display unit, 5903: operation switch, 5904: operation switch, 5905: band, 6100: expansion device, 6101: housing, 6102: cap, 6103: USB connector, 6104: substrate, 6106: controller chip, 7500: game machine, 7520: main body, 7522: controller

1. A data processing system comprising:
a biosensor;
a conversation data generation unit;
an operation unit;
a speaker; and
a microphone,
wherein the conversation data generation unit comprises a classifier that has learned first information of a user,
wherein the biosensor detects second information of the user,
wherein the conversation data generation unit generates first conversation data based on the first information and the second information,
wherein the speaker outputs the first conversation data,
wherein the microphone obtains second conversation data from the user and outputs the second conversation data to the classifier, and
wherein the classifier updates the first information with use of the second conversation data.

2. A driver assistance system comprising the data processing system according to claim 1, wherein the user is a driver.

3. A data processing device comprising the data processing system according to claim 1.

4. A data processing device comprising:
a conversation data generation unit;
an operation unit;
an image processing unit;
a display device;
an imaging device;
a biosensor;
a speaker; and
a microphone,
wherein the conversation data generation unit comprises a classifier that has learned first information of a user,
wherein the biosensor is configured to detect second information of the user who uses the data processing device,
wherein the imaging device is configured to capture a first image,
wherein the operation unit is configured to detect a designated first object in the first image,
wherein the image processing unit is configured to generate a second image where a second object overlaps with part of the first object when the first object is detected,
wherein the image processing unit is configured to display the second image on the display device,
wherein the conversation data generation unit is configured to generate first conversation data based on the first information and the second information,
wherein the speaker is configured to output the first conversation data in conjunction with movement of the second object,
wherein the microphone is configured to obtain second conversation data corresponding to a response from the user and output the second conversation data to the classifier, and
wherein the classifier is configured to update the first information with use of the second conversation data.

5. A data processing device comprising:
a conversation data generation unit;
an image processing unit;
a display device;
an imaging device;
an operation unit;
a biosensor;
a speaker; and
a microphone,
wherein the conversation data generation unit is supplied with first information of a user,
wherein the biosensor is configured to detect second information of the user who uses the data processing device,
wherein the imaging device is configured to capture a first image,
wherein the operation unit is configured to detect a designated first object in the first image,
wherein the image processing unit is configured to generate a second image where a second object overlaps with part of the first object when the first object is detected,
wherein the image processing unit is configured to display the second image on the display device,
wherein the conversation data generation unit is configured to generate first conversation data based on the first information and the second information,
wherein the speaker is configured to output the first conversation data in conjunction with movement of the second object,
wherein the microphone is configured to obtain second conversation data corresponding to a response from the user, and
wherein the conversation data generation unit is configured to output the second conversation data.

6. The data processing device according to claim 4, wherein the first information is preference information.

7. The data processing device according to claim 4, wherein the second information is biological information.

8. A wearable device comprising the data processing device according to claim 4, wherein the data processing device can serve as glasses.

9. A wearable device comprising the data processing device according to claim 4,
wherein the data processing device can serves as glasses, and
wherein a location where the second object is displayed can be designated by the user.

10. The data processing device according to claim 5, wherein the first information is preference information.

11. The data processing device according to claim 5, wherein the second information is biological information.

12. A wearable device comprising the data processing device according to claim 5, wherein the data processing device can serve as glasses.

13. A wearable device comprising the data processing device according to claim 5,
wherein the data processing device can serves as glasses, and
wherein a location where the second object is displayed can be designated by the user.
